SEQUENCE LISTING (56 sequences)

SEQ ID NO: 1 - protein, 201 aa, Solanum lysopersicum
MTKQDEGLTLELIRQHLLEDFTTTESFIDSLNSCFSDHISSSDDISPVFTSVKTEPSTSNSLSDSPNSSYPNEPNSPISRYFNLRSDFPEFKIDSDTILSPVFDSSAGSNEDNNKKKNYRGVRRRPWGKFAAEIRDPSRKGSRIWLGTFDTDIDAARAYDCAAFKMRGRKAILNFPLDAGKSGAPANVGRKRRRENKMELV

SEQ ID NO: 2 - protein, 222 aa, Solanum lysopersicum
MAPKEKIGAVTAMAMVNLNGISKEVHYRGVRKRPWGRYAAEIRDPGKKSRVWLGTFDTAEEAARAYDNAAREFRGAKAKTNFPKLEMEKEEDLKFAVKNEINRSPSQTSTVESSSPVMVDSSSPLDLSLCGSIGGFNHHTVKFPSSGGGFTGSVQAVNHMYYIEALARAGVIKLETNRKKTVDYLGGGDSDSSTVIDFMRVDVKSTTAGLNLDLNFPPPENM

SEQ ID NO: 3 - protein, 204 aa, Solanum lysopersicum
MARAQQRYRGVRQRHWGSWVSEIRHPLLKTRIWLGTFETAEDAARAYDEAARLMCGPRARTNFPYNPNMPQTSSSKLLSTTLTAKLHKCYMASLQMTKTSPQGQKLAKNATNVQESVINSYKMKQQMLVPKPSVLLTHHDHHEEAKVVNLGVGVIRKVEDQVLEGIPQFVKPLEDDHIEQMIEELLDYGSIELCSNVVPSHQIQ

SEQ ID NO: 4 - protein, 260 aa, Solanum lysopersicum
MCGGAILADIIPPRDRRLSSTDLWPTDFWPISTQNVPLNPKRARPSTGGEQMKKRQRKNLYRGIRQRPWGKWAAEIRDPRKGVRVWLGTFNTAEEAARAYDREARKIRGKKAKVNFPNEDDDHYCYSHPEPPPLNIACDTTVTYNQESNNCYPFYSIENVEPVMEFASYNGIEDGGEEMVKNLNNRVVEEEEKTEDEVQILSDELMAYESLMKFYEIPYVDGQSVAATVNPAAETAVGGGSMELWSFDDVSRLQPSYNVV

SEQ ID NO: 5 - protein, 193 aa, Solanum lysopersicum
MDYSSRDDLLFHYNSLPFNVNDTQDMLLYNLVAEGSSQETVNSSSSYGIKEEEVTSYEEERKDKNYRGVRKRPWGKYAAEIRDSTRNGVRVWLGTFDNAEEAALAYDQAAFAMRGSMAILNFPVEIVKESLNEMKCRFDGNCSPVIELKKRYSMRRKSVSRKNRARKDVVVFEDLGAEYLEELLISSESITNW

SEQ ID NO: 6 - protein, 234 aa, Solanum lysopersicum
MDQQLPPTNFPVDFPVYRRNSSFSRLIPCLTEKWGDLPLKVDDSEDMVIYGLLKDALSVGWSPFNFTAGEVKSEPREEIESSPEFSPSPAETTAAPAAETPKGRHYRGVRQRPWGKFAAEIRDPAKNGARVWLGTYETAEEAAIAYDKAAYRMRGSKAHLNFPHRIGLNEPEPVRVTAKRRASPEPASSSGNGSMKRRRKAVQKCDGEMASRSSVMQVGCQIEQLTGVHQLLVI

SEQ ID NO: 7 - protein, 314 aa, Solanum lysopersicum
MDSSSLEMIRQHLLDDVVFMETCSSSSSSSLETTSSTLYSQTSSNSESLESLTSEIKLESNFSVYPDFINTPQSSNLESVSRFFDNSTIEFQAKPQKKRSFNDRKPSLNISIPSVKKTEEPKTGEVKTGEPKTEEPKTGEVKTEYSVKEKMVENSEKKRYRGVRQRPWGKFAAEIRDPTRKGTRVWLGTFDTAMDAAMAYDRAAFRLRGSKAILNFPLEVSNFKQENHEIEKNVVNLNSNTNSCGKRVRGEMENDDGIVMKKEVKREQMVATPLTPSNWSSIWDCGNGKGIFEVPPLSPLSPHSNFGYSQLLVS

SEQ ID NO: 8 - protein, 244 aa, solanum lycopersicom
MGSPQETCTSLDLIRQHLFDESLDQTCFSFETTQTSNLDDIASFFNATSKTEYDGFFEFEAKRHVIRSNSPKQSNLRERKPSLNVAIPAKPVVVVENVEIEKKHYRGVRQRPWGKFAAEIRDPNRKGTRVWLGTFDTAVDAAKAYDRAAFKLRGSKAILNFPLEVANFKQQNDETKTEMKSSGSKRVRGETEELVIKKERKIEEERVLPTAAAPLTPSSWSTIWDEKGIFEVPPLSPLSQLVMI

SEQ ID NO: 9 - protein, 233 aa, Solanum lysopersicum
MESSSPKTQYPNFNFFQDQSSLPWNDQHFLDEYLTNIDQNNDHSLPGSTCSFLTSKESYRREVSSSNLHQLPRSWSSSNDTNSSKESNNRHEIEEVTSHHHDKNNSTKHYIGVRKRPWGKYAAEIRDSTRNGIRVWLGTFNTCEEAALAYDQAALTMRGPLALLNFPMDKVRESLENIKYICEDGISPAAVLKATNKMRRVKHKRNRKKRNVLVFEDLGAELLEELLTSTSSN

SEQ ID NO: 10 - protein, 264 aa, Solanum lysopersicum
MRHSLKMTTHHVENNNQEQDQVACEEILENVWANFISKNDQNSQKVTNEYCCEQYWEQLPILERLPSLGRWISMGAETWEDILNGIIIPSHNNENSNDESTCKDVVNVEKKEEKKKMVHYRGVRRRPWGKYAAEIRDSSRKGARVWLGTFSTAEEAAMAYDKAALRIRGPKAYLNFPHEMVAQAIGISNGPCEKEWTFSSSSQYNSRKRVSRDWNMYENLDEINQLPMEKKIMRSMEEDLFNDLDILEFEDLGSDYLDSLLSSL

SEQ ID NO: 11 - protein, 303 aa, Solanum lysopersicum
MCNIVHYKVANSNDNRSSRQDDEGINVFNTMFQGNINREEEMSVMVSALTRVVVGNHPSENIENHHQNNTLISRGVGEKRGRDEVLLHGTNSSHMILSSGGEGSSIRTTREATFIYTNSTNNSIIDESVNNQVRRRYRGVRQRPWGKWAAEIRDPYKAARVWLGTFDTAEGAARAYDEAALTFRGSKAKLNFPENVTLLVPSSIQQPIYSSPDPAISPYRSNFIIGHTSTEVEPILHTNPSNFIEPIAHTSSLYRSNFIERNHHMVQQEPYFQAGSTSGGSDFHQTTNSSNSSIYDHPSSSSG

SEQ ID NO: 12 - protein, 367 aa, Solanum lysopersicum
MCFLKVANSRKSSEFVRFTDTDDTQTTAVTAIGGGVEGGGQFDYSMYSGYCDSQARDMSEMVTEFTRVVSGQDYRPDTRCYSVNSPSPAYSSSSSGSRAGLKRSRDQQEFGTGLSSSSSVKIEEATSMVAPIPAFTTTITTTTTTGEGSSEETGGDRRRKYRGVRQRPWGKWAAEIRDPHKAARVWLGTFDTAEAAARAYDEAALRFRGNRAKLNFPENARLSSLPQTQNTVTSTISNPSPLIAQPTSFLNPIQSSDTTRDYWEYSQLLQNPGDFTDQQPSNLLEQMFVASSMAMLHSNTLPLISSSSSLATSATSSTSYPLLFSSYYTPQTNQIQGTNTSSTSTTSSSSFSTTFWSSSSQYPPSSS

SEQ ID NO: 13 - protein, 295 aa, Solanum lysopersicum
MHWLNKRFRQEAGMNSNSNSLQNNNQFQQQQPRLTGDEEYSVMVATLKNVINGNIPTQNYQEFNVFSPYNYSTATTTTNVTSSSSPSTSMSTSFEQVLGVSAEQEPCQFCRIQGCLGCDIFGTTFSSSSSAPAAVAAPVADNKKKSSSSSTATVAIAKKKKKNYRGVRQRPWGKWAAEIRDPRKAARVWLGTFTTAEEAARAYDKAAIEFRGPRAKLNFSFADYTVDTQEQQSTLSSSPQQLPEEPQQSQTANNNSDYGNEIWDQLMGDNEIQDWLTMMNFNGDSSDSGGNVHSF

SEQ ID NO: 14 - protein, 185 aa, Solanum lysopersicum
MSPPLFRVPEETERCQYCKINGCLGCNYFATSSAAAGVVNNNKALKIVGKTKKKKKNYRGVRQRPWGKWAAEIRDPRRAARVWLGTFTTAEDAARAYDRAAIEFRGPRAKLNFSFTDYTSIQQHNTTTPMQVLQQQQPAPSQLQQGINTEEEEFWDQLMNSDNEIQHYLYRESSDSANGYIAHSF

SEQ ID NO: 15 - protein, 254 aa, Solanum lysopersicum
MCGGAIISDYDPAGSFYRKLSARDLWAELDPISDYWSSSSSSSTVGKPDSALSPVTHSVDKPNKSDSGKKGNKTVKVEKEKSSGPRPRKNKYRGIRQRPWGKWAAEIRDPQKGVRVWLGTFNTAEDAARAYDEAAKRIRGDKAKLNFPAPSPPAKRQCTSTVAAADTPPALLLESSDNSPLMNFGYDVQYQSQTPYYPMEMPIVSEDYELKEQISNLESFLELEPSDQFSGIVDSDPLNVFLMEDFASTHHQFY

SEQ ID NO: 16 - protein, 221 aa, Solanum lysopersicum
MRRGRATPAAAAAAVKPDGSGGLKEIRFRGVRKRPWGRFAAEIRDPWKKTRVWLGTFDSAEDAAKAYDAAARTLRGPKAKTNFPLPMYSQHHQFNRSLNPNDRLVDPRLYSQEAPIICQRPTSSSMSSTVESFSGPRPPRQQTAVLPSRKHPRSPPVEPDDCRSDCDSSSSVVEDGDCEGGNDNIVSSSLRNPLPFDLNFPPPMDDVYANSNDLYCTALCL

SEQ ID NO: 17 - protein, 240 aa, Solanum lysopersicum
MRRSRAAAAARQVPATEVPVPAPVAGEHNGSGGSKEIRFRGVRKRPWGRFAAEIRDPWKKTRVWLGTFDSAEDAARAYDAAARTLRGPKAKTNFPLPSSHHLPPYPHHHQFNQSINPNDPFVDSRLYSQDHPLVSQRPTSSSMSSTVESFSGPRQPPRQQTAASVPSRKYPRSPPVVPDDCHSDCDSSSSVVEDGECDNDNIASSSFRKPLPFDLNLPAPMDDFSADAYADDLHCTALCL

SEQ ID NO: 18 - protein, 198 aa, Solanum lysopersicum
MRHRKSSELKRPGSDLLQQPDADPPRYRGVRKRPWGRFAAEIRDPIKKTRVWLGTFDTAEDAARAYDDAARALRGAKAKTNFNMLPLTDDPYDDEFELFPNPRPASSSMSSTLESSSGPRGGSSSKVTRMKIPRPVRPMEECRSDCDSSSSVVDDRCDVDQTSSFVTKQPLPFDLNLPPPSDNDGVDVDDLHVTALCL

SEQ ID NO: 19 - protein, 225 aa, Solanum lysopersicum
MAVKDKAVKGGNVKVNHGVKEVHYRGVRKRPWGRYAAEIRDPGKKSRVWLGTFDTAEEAAKAYDAAAREFRGPKAKTNFPFPAEMNNVGNNNSQSPCGSSTVESSSGETVVHAPNTRHAPLELDLTRRLGAAAEGGRGGVGYPILHQQPTVAVLPNGQPVLLFDSMWRPGVVSRPYQVVPATMEFAGVGAGVVTSVSDSSSVVEEKHYGKKGLDLDLNLAPPMEV

SEQ ID NO: 20 - protein, 293 aa, Solanum lysopersicum
MESQKIKKKLVHKTITTKYDHHNKWTPKVVRICYTDCDATDSSSDDDDDERNRVKKYVTEIKFEKKMAAADVRKSLNSNKKKKKAIDLKRDENVKKFRGVRQRPWGKWSAEIRDPVKKTRVWLGTFDTAEEAAMKYNIAAIQLRGADAIINFIETPFPKENAITSVSDYDSTGECENLCSPTSVLRQNNNNNDKDNEDAIAIDTKIMNDESKKMEMDENGFMFDDNLPLMDQSFLKDFFDFRSPSPLMDDVLLPGFSDGMGLLPEVLSIHGNRMLDEDLETCKWANDFFQDVC

SEQ ID NO: 21 - protein, 242 aa, Solanum lysopersicum
MYQLPTSTELTFFPAEFPVYCRSSSFSSLMPCLTESWGDLPLKVNDSEDMVIYGFLQDAFSIGWTPSNLTSEEVKLEPREEIEPAMSTSVSPPTVAPAALQPKGRHYRGVRQRPWGKFAAEIRDPAKNGARVWLGTYESAEEAALAYDKAAFRMRGTKALLNFPHRIGLNEPEPVRVTVKRRLSESASSSVSSASESGSPKRRRKGVAAKQAELEVESRGPNVMKVGCQMEQFPVGEQLLVS

SEQ ID NO: 22 - protein, 224 aa, Solanum lysopersicum
MSSPLEIDTSFSHSNLLFLEDESSWSNTHDPFVDIDEYLPIIIPCNDEEIVVESSNTSTTTTTTTTSKVASIQNIHHDQEEVTSIEKKHEDDQEKHYIGVRKRPWGKYASEIRDSTRNGIRVWLGTFDTAEEAALAYDQAALSMRGPWSLLNFPMEHVKKSLENIEYSCKDGLSPAAVLKATHKTRRVKHKRSSRKKKNENLENVFVFQDLGVELLEELLMTSS

SEQ ID NO: 23 - protein, 240 aa, Solanum lysopersicum
MDSSSSSSQFFYSMNSDLNSSDSSYEWSNFNTQSYLPFNVNDSEEMLLFGVLNAAHEETTSETVTSHRVKEEEVTSESEVIEAIPAKEKSYRGVRRRPWGKFAAEIRDSTRNGVRVWLGTFDSAEDAALAYDQAAFSMRGNSAILNFPVETVRDSLRDMKCHVDDDCSPVVALKKRHSMRKRSTNSKKVNSISKVVREVKMENVNNVVVFEDLGADYLEQLLSSSSSDQSSCDATYFSPW

SEQ ID NO: 24 - protein, 161 aa, Solanum lysopersicum
MVPTPQSDLPLNENDSQEMVLYEVLNEANALNIPYLPQRNQLLPRNNILRPLQCIGKKYRGVRRRPWGKYAAEIRDSARHGARVWLGTFETAEEAALAYDRAAFRMRGAKALLNFPSEIVNASVSVDKLSLCSNSYTTNNNSDSSLNEVSSGTNDVFESRC

SEQ ID NO: 25 - protein, 372 aa, Solanum lysopersicum
MCGGAIISDLVPPSRISRRLTADFLWGTSDLNKKKKNPSNYHSKPLRSKFIDLEDEFEADFQHFKDNSDDDDDVKAFGPKSVRSGDSNCEADRSSKRKRKNQYRGIRQRPWGKWAAEIRDPRKGIRVWLGTFNSAEEAARAYDAEARRIRGKKAKVNFPDEAPVSVSRRAIKQNPQKALREETLNTVQPNMTYISNLDGGSDDSFSFFEEKPATKQYGFENVSFTAVDMGLGSVSPSAGTNVYFSSDEASNTFDCSDFGWAEPCARTPEISSVLSEVLETNETHFDDDSRPEKKLKSCSSTSLTVDGNTVNTLSEELSAFESQMKFLQIPYLEGNWDASVDAFLNTSAIQDGGNAMDLWSFDDVPSLMGGAY

SEQ ID NO: 26 - protein, 327 aa, Solanum lysopersicum
MCGGSIISDYIDPSRTSRRLTAEFLWGRFDLGKKQKNPNNYHSKAKHLRSEVVDDFEADFQDFKELSDDEDVQVDVKPFAFSASKHSTGSKSLKTVDSDKDAAADKSSKRKRKNQYRGIRQRPWGKWAAEIRDPRKGVRVWLGTFNTAEEAAKAYDIEARRIRGKKAKVNFPDEAPAPASRHTVKVNPQKVLPEESLYSLQSDSAIMNSVEDDHYDSFGFFEEKPMTKQYGYENGSSASADTGFGSFVPSAGGDIYFNSDVGSNSFECSDFGWGEPCSRTPEISSVLSAAIECNEAQFVEDANSQKKLKSCTNNPVADDGNPRYYGT

SEQ ID NO: 27 - protein, 248 aa, Solanum lysopersicum
MTENSVPVIKFTQHIVTTNKHVFSEHNEKSNSELQRVVRIILTDADATDSSDDEGRNTVRRVKRHVTEINLMPSTKSIGDRKRRSVSPDSDVTRRKKFRGVRQRPWGRWAAEIRDPTRGKRVWLGTYDTPEEAAVVYDKAAVKLKGPDAVTNFPVSTTAEVTVTVTETETESVADGGDKSENDVALSPTSVLCDNDFAPFDNLGFCEVDAFGFDVDSLFRLPDFAMTEKYYGDEFGEFDFDDFALEAR

SEQ ID NO: 28 - protein, 234 aa, Solanum lysopersicum
MYSNCELENDFSVLESIRRYLLEDWEAPLTSSENSTSSEFSRSNSIESNMFSNSFDYTPEIFQNDILNEGFGFGFEFETSDFIIPKLESQMSIESPEMWNLPEFVAPLETAAEVKVETPVEMTTTTTKPKAKHYRGVRVRPWGKFAAEIRDPAKNGARVWLGTYETAEDAALAYDKAAFRMRGSRALLNFPLRINSGEPDPVRVGSKRSSMSPEHCSSASSTKRRKKVARGTKQ

SEQ ID NO: 29 - DNA, 606 bp, Solanum lysopersicum
atgacgaaacaagatgaaggattaacattagaactcatacgacaacatctcctcgaagatttcacaactacagaatcattcatcgacagtctcaattcttgtttttccgatcacatctcctcctccgatgacatctcccctgttttcacttcagtaaaaacagagccatctacatccaattccctctcagattcacccaattcctcatacccaaatgaacccaactccccaatttcccgttacttcaatctccgctccgatttccctgaattcaaaatcgattcagataccatcctcagtccagttttcgacagctccgccggttctaatgaagacaataataagaagaagaattacagaggggtaaggagaaggccatgggggaaatttgcggcggagataagagatccaagtcgaaaaggatcgaggatttggttgggtacttttgatactgatattgatgctgctagagcttatgattgtgcagcgtttaagatgagaggaagaaaagctattctgaattttccgttggatgccggaaaatctggtgctccggcgaatgttggccggaaaaggagaagagagaacaagatggagttggtgtag

SEQ ID NO: 30 - DNA, 669 bp, Solanum lysopersicum
atggcgcctaaggaaaaaattggtgcagttacagctatggcaatggtgaatttaaatggaatttcgaaagaggtgcattatagaggtgtaaggaagaggccatgggggagatacgcggcggagattagagatcctgggaaaaaaagtagggtttggttaggtactttcgatactgcggaggaggcggctagagcttatgataacgctgctagagaatttcgtggagcgaaagcgaaaactaattttccgaaattagaaatggaaaaagaggaagatctgaaattcgctgtgaaaaatgaaatcaatcggagtccgagtcagactagtactgtggagtcatcgagtccggttatggttgattcatcatcgccgttagatctaagtctctgtggatcaatcggcgggtttaatcatcatacggttaagttcccgagctccggtggaggttttaccggttcggtacaggcggtgaatcatatgtactatatagaagcacttgcacgcgccggagttataaagttagaaacaaatcggaagaaaacggtagattacctcggtggtggtgactctgattcatcaacggtaattgattttatgcgtgttgacgtgaaatcaaccaccgccggtttaaatctggatctcaactttcctccaccggaaaacatgtga

SEQ ID NO: 31 - DNA, 615 bp, Solanum lysopersicum
atggctagggcacaacaaagatatcgaggagttcgacagagacattggggttcttgggtctccgaaattcgccatccattgttgaagacaagaatttggttaggcacttttgagacagcagaagatgcagcaagagcatatgatgaagcagcaaggctaatgtgtggtccaagagctagaactaatttcccatacaacccaaacatgccacaaacatcttcctctaagctactctcaactacattaacagccaagttacacaaatgctacatggcttcacttcaaatgaccaaaacctcaccacaaggacaaaaattagcaaaaaatgcaaccaatgttcaagaaagtgttattaattcctataaaatgaaacaacaaatgttggtaccaaagccatcagtactattgactcatcatgatcatcatgaggaagctaaagtagtcaacttgggagtgggagtaattaggaaagttgaagatcaagtacttgagggtataccacaatttgtcaagccacttgaagatgatcacattgaacaaatgattgaagaattgttggattatggatccattgagctttgctctaatgttgttccttctcaccaaatccagtga

SEQ ID NO: 32 - DNA, 783 bp, Solanum lysopersicum
atgtgtggtggtgcaattcttgctgatatcattcctcctcgtgaccgccgtttgtcatccaccgacctatggccgactgatttctggccaatttccacccaaaatgttcctctcaaccccaaacgagctcgaccctctacaggtggtgagcagatgaagaagaggcaaaggaagaatctttacagagggataagacaacgtccatggggtaaatgggctgctgaaattcgtgacccgagaaaaggggttagggtttggttaggtactttcaacactgctgaagaagctgcaagagcttatgatagagaagctcgtaaaatcaggggtaagaaagctaaagttaatttccccaatgaagatgacgaccattactgctacagtcatccagagccccctcccttgaacattgcttgtgatactactgttacttacaatcaagaatcaaataactgttaccccttttactcaatcgagaacgttgaacctgttatggaatttgcaagttataatggaattgaagatggaggagaggagatggtgaaaaatttgaataacagggttgtagaggaagaggagaaaacagaggatgaagtgcagatactttctgatgagctgatggcttatgagtcattgatgaagttctatgaaataccgtatgttgacgggcaatcagtggcggcgacggtgaatccagcggcggagaccgccgtgggcggtggctcgatggagctttggagttttgatgatgttagtcgtctacaaccaagttataatgtagtttaa

SEQ ID NO: 33 - DNA, 582 bp, Solanum lysopersicum
atggattattcatctcgggatgatcttctttttcactataattcacttccatttaacgttaacgatacacaagacatgttactttataatcttgttgctgaaggatcatcgcaagaaacagtgaattcgtcgtctagctatggaataaaggaagaggaagtgacctcatatgaagaagaaagaaaagataagaattacagaggtgttagaaagaggccatggggtaaatatgctgctgaaattcgtgattctacgaggaatggtgttcgtgtatggctaggtacatttgataatgctgaagaagctgcgttagcgtatgatcaagctgcatttgctatgagaggttcgatggccatacttaattttcctgtggagatagttaaggaatcgctaaatgaaatgaagtgtagatttgatggtaattgttcgccggtgattgaattgaagaagagatattcaatgaggagaaagagtgttagtagaaaaaacagagcaagaaaagatgttgtggtttttgaagatttgggtgctgagtatttagaggaattgttgatttcttcagaaagtatcacaaattggtga

SEQ ID NO: 34 - DNA, 705 bp, Solanum lysopersicum
atggatcaacagttaccaccgacgaacttcccggtagattttccggtgtatcgccggaattcaagcttcagtcgtctaattccctgtttaactgaaaaatggggagatttaccactaaaagtcgacgattccgaagatatggtaatttacggtctattaaaagacgctctaagcgtcggatggtcgccgtttaatttcaccgccggcgaagtaaaatcggagccgagagaagaaattgaatcgtcgcctgaattttcaccttctccggcggagaccacggcagctccggcggctgaaacaccgaaaggaagacattatagaggcgttagacagcgtccgtgggggaaatttgcggcggagattagagatccggcgaagaacggagctagggtttggcttggaacgtacgaaacagctgaagaagctgcaattgcttatgataaagctgcttatagaatgagaggatcaaaagcacatttgaatttcccgcaccggatcggtttgaatgaaccggaaccggttcgagttacggcgaaaaggcgagcatcgccggaaccggcaagctcgtcgggaaacggttccatgaaacggagaagaaaagccgttcagaaatgtgatggagaaatggcgagtagatcaagtgtcatgcaagttggatgtcaaattgaacaattgacaggtgtccatcaactattggtcatttaa

SEQ ID NO: 35 - DNA, 945 bp, Solanum lysopersicum
atggattcttcttcactagaaatgataagacaacatcttcttgatgatgttgttttcatggaaacttgttcttcttcttcttcttcttcattagaaacaacaagtagtacactttattctcaaacctcatcgaattcggaatctttagaatcattaacctctgagatcaaacttgaaagcaatttctctgtttatcctgatttcatcaatacacctcaaagttcaaatcttgaatctgtctctcgttttttcgataactcaactattgaattccaagctaaaccccaaaagaaaagaagtttcaatgatcgaaaaccttcgttaaacatttcgattccttctgttaagaaaacagaggaaccaaaaacaggggaagtaaaaacaggggaaccaaaaacagaggagccaaaaacaggggaagtgaaaacagagtactctgttaaggagaaaatggtagaaaattcggagaaaaagcgatacagaggagtgagacaaaggccatgggggaaatttgcagcggagattcgtgacccaactagaaaggggacacgagtttggttaggaacattcgatactgcaatggatgcagccatggcatatgacagagcagcatttaggctcagagggagtaaagcaatcttgaattttccacttgaggtaagcaatttcaagcaagaaaatcatgagattgagaaaaatgttgtgaatttgaattcgaatacgaattcttgtgggaaaagggtgagaggggaaatggagaatgatgacggaattgtaatgaagaaagaggtgaaaagagaacaaatggtggcaactccattaacaccttcaaattggtcttcaatttgggattgtggaaatggaaaaggtatttttgaagtgccacctttgtcaccattatcaccacattcaaattttggttattctcaacttttggtatcatag

SEQ ID NO: 36 - DNA, 735 bp, Solanum lysopersicum
atgggttctccacaagagacttgtacttcacttgatttgattaggcaacatctttttgatgaatctctggaccagacttgtttctcttttgaaacaactcaaacttcaaatcttgatgacatcgcaagcttctttaatgctacttcaaaaacagagtatgatggttttttcgaatttgaggcaaaacgacatgttatccgttcaaattctccgaaacagagtaacttgagagaacggaagccatctctgaacgtagcaataccggcgaagcctgttgttgttgtagagaacgttgagattgagaagaagcattacaggggagttagacagaggccatgggggaagtttgcagcggagattcgtgacccaaatagaaaggggactcgagtttggttaggaacatttgatactgctgtggatgcggcaaaggcatatgacagggcagcgtttaagcttagaggaagcaaagcaatattgaatttcccactcgaagttgcaaactttaagcaacaaaatgatgagactaaaacagagatgaagtcgtcaggcagtaaaagggtgagaggagaaacagaggaattagtaatcaagaaggaaaggaaaatagaagaagaaagagtactcccaacggcggcggctccattaacaccgtcaagttggtcgacgatttgggatgaaaagggtatttttgaggtgccaccattgtcaccattatctcagcttgttatgatataa

SEQ ID NO: 37 - DNA, 702 bp, Solanum lysopersicum
atggaatcttcatcccctaaaactcaatatccaaatttcaatttcttccaagatcaatcatcattaccatggaatgatcaacatttcttagatgaatatttgactaacatcgaccaaaacaacgatcattctctaccaggaagtacttgttcattcttaacctcgaaagaaagttatagacgggaagtttcctcctccaacctacatcaattaccaagaagttggtcatcctcaaacgatacgaattcctctaaagaaagcaataatcgtcatgaaatcgaagaggtcacgtctcatcaccatgataagaataactccaccaaacactacataggagttagaaagagaccatggggaaaatatgcagcggaaataagggattcaacaagaaatgggattagggtttggttaggaacatttaatacttgtgaagaagctgctttagcgtatgatcaagctgcacttacaatgagaggtccattggcacttttaaattttccaatggacaaagtaagagaatcacttgaaaatattaagtatatttgtgaagatgggatttcaccagctgctgttttaaaggcaacaaataaaatgagacgtgttaaacataagagaaataggaagaagagaaatgttttggtttttgaagatttgggtgctgaattgttagaggaacttttgacgagtacttcctctaattag

SEQ ID NO: 38 - DNA, 795 bp, Solanum lysopersicum
atgagacattctttaaagatgactactcatcatgtggaaaataataatcaagaacaagatcaagtagcatgtgaagagattctagagaatgtttgggcaaacttcatatccaagaatgatcaaaattctcaaaaggtgacaaatgaatattgttgtgaacaatattgggaacaacttccaattcttgaaagattaccaagcttaggaagatggatatcaatgggagctgaaacttgggaagatattctcaatggaattattattccttctcataacaacgaaaattcaaacgacgaatcaacatgcaaagatgttgtcaacgttgagaagaaggaggagaagaagaagatggtgcattatagaggggtaagaaggaggccatggggaaaatacgcggctgagataagggattcatcaagaaaaggagctagggtttggctagggacatttagtactgctgaagaagctgctatggcttatgacaaggcggctttgagaattagaggtcccaaggcctatcttaattttccacatgaaatggttgctcaagctataggaatatctaatggtccttgtgaaaaagaatggaccttttcatcatcaagtcaatataattcaaggaaaagggtatcaagggattggaacatgtatgaaaatttggatgaaattaatcaattgcctatggagaagaaaattatgagaagcatggaggaggatctttttaatgacttggatatcctagagtttgaggatcttggaagtgattatttggatagtttattatcctctttgtaa

SEQ ID NO: 39 - DNA, 912 bp, Solanum lysopersicum
atgtgtaatattgttcactacaaggtggcgaattcgaatgataacaggagtagtagacaagacgatgaagggattaatgtgtttaatacgatgtttcaagggaatattaatagagaagaagaaatgtctgttatggtttctgcattaactcgtgttgttgttggtaatcatcctagtgaaaatatcgaaaatcatcatcaaaataatacattgatttctaggggtgttggagaaaaaagaggacgtgatgaagtattattacatggaactaattcttctcatatgatattatcatcaggtggtgaaggttcaagcattaggacaacaagagaagcaacattcatatacactaattcaacaaacaatagcattattgatgaatctgttaataatcaagtaagacgacgatacagaggagttagacagaggccatgggggaaatgggcagctgaaataagagatccatataaagcagcgcgtgtttggttaggtacttttgatactgctgaaggggctgctagagcttatgatgaagctgctcttacatttagaggtagtaaagcaaaactaaatttcccagaaaatgttacattattagtgccttcttcaattcaacaacccatttactcctcacccgacccagccatttctccgtatcgttccaattttattattggacatacctctacggaagtggagcctatacttcacaccaatccttcaaattttattgagcctatagctcacacttcatctctgtatcgttccaattttattgaacggaatcatcacatggtgcagcaggagccgtattttcaggcaggtagtactagtggtggaagtgattttcatcaaactacaaattcttctaattcgtcaatttatgatcacccttcttcttcatctggataa

SEQ ID NO: 40 - DNA, 1104 bp, Solanum lysopersicum
atgtgctttttaaaggtggcgaattcaagaaaatcaagtgaatttgttagatttacagatacagatgatacacaaaccaccgccgtgactgcgatcggcggcggtgttgaaggcggtggtcagtttgattattcgatgtattcagggtattgtgattctcaggcgagagatatgtcggagatggtgacggagtttacacgtgtggtatcgggtcaggattatcgacccgataccagatgttattcggttaattcaccgtctccggcttattcttcgtccagctcgggttctagagctggactgaagagaagccgtgatcaacaagaatttggaactgggttgtcatcttcttcctctgttaaaattgaagaagctacaagtatggttgcaccaattcccgctttcacaaccacaatcacaaccacgaccacaacaggtgagggttcgagcgaagaaacaggaggagataggaggaggaaatacagaggtgtacgacaacgaccatggggtaaatgggcagcggaaataagagatccacataaagccgccagagtttggttaggaacattcgatacagcagaagctgcagcaagagcatatgatgaagctgcattgagatttcgaggaaacagagcaaaactcaacttccctgagaacgccagattgtcatcgttaccacaaacacaaaatactgtaacgtcaacaatctccaatccatcccctctaatagctcaaccaacgtcgttcctcaatcctatccagagttcagatacaacaagagactactgggaatactcacaattgttgcaaaatccaggagattttacggatcaacaaccatcaaacttattggaacaaatgttcgttgcctcatcgatggcaatgttgcattcaaacacattgccattaatatcttcgtcttcatcgttagctacatcagcaacttcttcaacgtcatatcccctgttattttcgagttattacacaccacaaactaatcaaattcaaggaaccaacacaagtagcaccagcaccactagcagctcaagtttttctacaacattttggagtagctctagccaatatcctccatcttctagttaa

SEQ ID NO: 41 - DNA, 888 bp, Solanum lysopersicum
atgcattggttaaataaaagatttagacaagaagcaggaatgaattcgaattcgaattccctccaaaataacaatcaatttcaacagcagcaaccaaggcttactggagatgaagagtactctgttatggttgcaactctgaaaaatgtgatcaatggtaatattccaacgcaaaattatcaagaattcaatgtcttttcgccatataattattctactgccaccacaaccacgaatgttacttcttcttcttcgccttctactagtatgtctactagtttcgaacaagtattgggtgtctctgctgaacaagaaccttgtcaattttgcagaattcaaggttgtttaggctgtgacatttttggtaccacattttcttcttcttcttcagcgcctgctgctgtggctgctcctgttgctgataataagaagaagagtagtagtagtagtacagctacagtcgcaatcgcgaagaaaaagaagaagaattacagaggagtgagacagaggccatgggggaaatgggcagcagaaattcgtgatcctcgaaaagctgcacgtgtttggctgggaactttcactacggcagaagaagcagctagagcttatgataaagccgccattgaattcaggggtccacgagctaaattgaatttttcatttgcggattataccgttgacactcaagaacaacagagcactttatcttcttcaccacaacaattaccagaagagcctcagcaatcccagacagcgaataataattccgattatggaaatgaaatttgggatcaattgatgggtgacaatgaaattcaagattggttgaccatgatgaatttcaatggcgactcttctgattctggcgggaatgttcacagcttttaa

SEQ ID NO: 42 - DNA, 558 bp, Solanum lysopersicum
atgtcgccgcccttgtttcgcgtaccggaagaaacagagcgttgtcagtactgcaaaataaatggttgtttaggctgcaactattttgcaacctcatcagctgcagctggtgttgttaacaacaacaaggcattaaagattgttgggaagacgaaaaagaagaagaagaattacaggggagtgagacagagaccatggggaaaatgggcagcggaaattagagatccaagaagggcagctagagtatggcttggaacatttactacagctgaggacgcagctagagcttatgacagagcagctattgaatttagaggtccaagagctaagcttaatttctcatttacagattacacttcaattcaacaacacaatactactacacccatgcaagtgctgcaacaacaacaaccagctccctcgcagttacaacaaggaataaacacagaagaagaagagttctgggatcaattgatgaattcggacaatgaaattcaacattatctttatagagaatcatctgattctgctaatggctatattgctcatagcttctag

SEQ ID NO: 43 - DNA, 765 bp, Solanum lysopersicum
atgtgtggaggtgccataatctccgattatgatcccgccggaagcttctaccggaaactttctgctcgtgacctctgggctgagctggaccctatctccgactactggtcctcttcttcctcatcctcaaccgtcggaaaacctgattccgctctgtcgccggtgactcactccgtcgataagccaaataaatcagattccggcaaaaaaggtaataagactgtgaaggttgagaaggagaagagtagtggaccaaggccaaggaagaacaagtacagaggaataagacagaggccatggggaaaatgggctgctgagattcgcgatccacagaagggtgtacgcgtttggcttggtacattcaacacagcagaagatgctgctagagcctatgatgaggctgctaagcgcattcgtggtgataaggctaaactcaactttccagccccatcaccaccagctaagcgacagtgcactagcactgtcgctgctgctgatacaccaccagcactactccttgagagttctgacaactctcctttgatgaactttggatatgatgtccagtatcagagccaaactccctactaccccatggaaatgcccatagttagtgaagattatgaactgaaggaacagatttccaatttggaatcgttcctggaattggagccatctgatcaattttcagggatcgtcgattctgatcctcttaatgtttttctgatggaggactttgcttcaactcatcatcagttctactga

SEQ ID NO: 44 - DNA, 666 bp, Solanum lysopersicum
atgagaagaggcagagcaactccggcggcggcggcggcggcggtgaagccagatggatctggaggattgaaggagattaggtttcgtggagttcggaagaggccatgggggagatttgctgcagagattagagatccatggaagaaaactagggtttggttaggtacttttgattcagctgaagatgctgccaaagcttatgatgctgcagctcgaactcttcgtggacctaaagctaaaactaatttccctttacctatgtattctcagcatcatcagttcaatcgaagtttaaaccctaatgatcggttagttgacccgagattgtactcacaagaagctccgatcatttgtcaaagacctacatcgagcagtatgagtagtactgtggaatcattcagtggaccgagaccgccacgtcagcaaacggcggttttgccttcgagaaaacatcctagatcgccgccggttgagccggatgactgcaggagtgattgtgactcatcgtcttctgttgttgaagatggtgattgtgaagggggaaatgacaacatcgtttcttcatctctcagaaatccactgcctttcgatctcaactttccacctccgatggatgatgtttatgctaattcaaatgatctttactgcacagcactatgtctttga

SEQ ID NO: 45 - DNA, 723 bp, Solanum lysopersicum
atgcggagaagcagagcagccgctgcggcgagacaagttccggcgacggaagttccggtaccggcaccggtggccggagaacacaacggatctggaggatctaaggagataaggttccgtggagttcgaaagagaccatggggaagatttgcagcagagattagagatccatggaagaaaactagggtttggttgggtacttttgattctgctgaagatgctgctcgtgcttatgatgcagcagctcgtactcttcgtggacctaaagctaaaactaatttccctttaccttcttctcatcatctacctccatatcctcaccatcatcagttcaaccaaagcatcaaccctaacgatccctttgtcgattcccggttatactctcaggaccacccattagtttcacagagacctacttcaagcagcatgagtagtacggtggagtccttcagtggaccacggcagccgccgcggcagcagacggcagcttccgtgccttccagaaagtatccccggtcaccgcctgttgtcccggacgattgccatagcgactgtgactcatcgtcttctgtcgttgaagacggtgaatgtgataacgacaacatcgcttcttcctctttcagaaagccgttgcctttcgatctaaacttaccggcaccgatggatgacttcagcgccgacgcatatgccgatgatcttcactgcacagcactatgtctttga

SEQ ID NO: 46 - DNA, 597 bp, Solanum lysopersicum
atgcgccaccggaagtcgtcggagctgaaaagaccaggatctgacctccttcaacagcctgacgccgacccacctcgttatcgaggtgttcgtaaacggccatggggtagattcgcagcagagattagagatccgattaaaaagactcgagtttggctgggtacctttgacacagctgaagacgccgcacgcgcttacgacgatgctgcacgcgctctccgtggagctaaggcgaaaactaatttcaatatgttacctctaacagatgatccttatgatgatgagtttgagcttttccccaatccgagaccggcttctagcagtatgagcagtacgttggaatcgtctagtgggcctcgtggcggatcgagtagtaaggtgacccggatgaagattcctcgcccagttcgtccgatggaggaatgccggagtgattgcgattcgtcgtcgtctgtggtggatgatcggtgtgatgttgatcaaacgtcatcgtttgtgaccaaacaacctctgccgttcgatctgaatctgccacctccgtcggataacgatggagttgatgttgatgatttgcacgtcaccgctttatgcctctaa

SEQ ID NO: 47 - DNA, 678 bp, Solanum lysopersicum
atggctgtgaaagataaggctgtgaaaggaggtaatgtgaaagtgaatcatggagttaaggaagttcactacagaggtgtaaggaagaggccatggggtcgttacgctgcggagattcgtgacccgggtaagaagagtcgggtctggctgggtacttttgatacggcggaggaagcggctaaggcttacgatgccgctgccagagagtttcgtggacctaaagcgaagacgaatttcccctttccggcggagatgaataatgttggtaacaataacagtcagagcccgtgtgggagcagtaccgtggagtcatccagcggagaaacggttgttcacgcgcctaatacgcgacacgcgccgctggagctggatctcacgcgccgtctcggtgccgctgctgaaggtggacgtggaggtgtcggctacccgatcttacaccagcagccgacggtggcggttctgccgaacggtcagccggttttgttgtttgattctatgtggagaccgggagttgttagtaggccgtatcaggttgtaccggcgacgatggagtttgccggtgtcggtgccggagttgttactagtgtgtcggattcgtcttccgttgtggaagagaaacattatgggaaaaagggacttgatcttgatcttaaccttgcgccacctatggaagtttaa

SEQ ID NO: 48 - DNA, 882 bp, Solanum lysopersicum
atggaatcacaaaaaatcaaaaagaaattagtccacaaaactatcactactaagtatgatcatcacaacaagtggactcctaaagttgttcggatttgttacactgattgtgatgctactgattcttcaagcgacgacgatgacgacgagaggaatcgagtgaaaaaatacgttacagagattaaatttgagaagaaaatggctgctgcagatgtgaggaaatcgttgaattcgaataagaagaagaagaaagcgatcgatttgaagagagatgagaatgttaaaaagtttcgcggtgtcagacagaggccatggggaaaatggtctgcggagattcgagatccggtgaaaaaaacgagggtttggttaggtacttttgataccgctgaagaagcggctatgaaatataatatagccgctattcaattgcgcggagctgatgctatcattaattttattgagacacctttcccaaaggaaaatgcgatcacttcagtatcggattatgattccacaggggaatgtgaaaacctctgttctcccacctcagttttgaggcagaataataacaataatgataaagataacgaagatgcgattgcgattgatactaaaattatgaacgatgagagcaaaaaaatggaaatggatgaaaatggatttatgtttgatgataatttgccattaatggatcagagtttccttaaggatttcttcgattttcgatccccttctccattgatggatgatgtattattaccaggttttagcgatggaatgggattattaccagaagtgttgagtattcatggaaatagaatgttggatgaagatttggagacttgtaagtgggcaaatgatttcttccaagatgtttgttga

SEQ ID NO: 49 - DNA, 742 bp, Solanum lysopersicum
atgtatcaacttcccacttctactgagttaactttttttccggcagaattcccggtgtattgccggagttcaagtttcagtagtctcatgccatgtttaaccgaatcatggggtgacttgccgttaaaagttaacgattccgaagatatggtaatttatgggtttctacaagacgcttttagtatcggatggacgccgtcaaatttaacgtccgaggaagtgaaactcgagccgagggaggagattgagccagctatgagtacttctgtttctccgccgacagtggctccagcggctttgcagcctaaaggaaggcattacaggggcgttagacaaaggccatggggaaaatttgcagcggaaataagagatccggctaaaaacggcgcacgggtttggcttggaacttacgagtcggctgaggaagccgcactcgcttatgataaagccgcttttaggatgcgcggtactaaggctctattgaatttcccgcatagaattggtttaaatgagccggagccggttagagtgacggttaagagacgattatctgaatcggctagttcatcggtatcatcagcttcggaaagtggctcgcctaagaggaggagaaagggtgtagcggctaagcaagccgaattagaagttgagagccggggaccaaatgttatgaaagttggttgccaaatggaacaatttccagttggcgagcagctattggttagttaaaatatggagctaa

SEQ ID NO: 50 - DNA, 675 bp, Solanum lysopersicum
atgtcaagcccactagagatagatacttcattttcacattccaatttgttgtttttggaagatgaatcatcatggagtaatactcatgatccatttgttgatattgatgaatatctaccaataattataccatgtaatgatgaagaaatagtagtagaatcctcaaacactagtactacaacaacaacaacaacaacatcaaaagtagcaagtatccaaaatattcatcatgatcaagaagaggtaacatccatagagaaaaaacatgaagatgatcaagaaaaacattatattggagttagaaagaggccatggggtaaatatgcatcagaaattagggattcaacgcgtaatggaattagggtttggttaggaacatttgatactgctgaagaagctgctttagcttatgatcaagccgcattatcaatgaggggtccttggtctctactcaattttccaatggaacatgttaaaaaatctcttgaaaatattgagtattcttgtaaagatggattatctccagctgctgttttaaaagctactcataaaactagaagagtcaagcacaagagaagtagtagaaagaagaagaatgagaatttggaaaatgtttttgtttttcaagacttgggagttgaattattagaagagcttttaatgacttcatcatag

SEQ ID NO: 51 - DNA, 723 bp, Solanum lysopersicum
atggattcttcttcttcttcatctcaattcttctactcaatgaattctgatttaaattcatcagattcttcatacgaatggtccaatttcaacacacaatcttatctccctttcaacgtgaacgactccgaagagatgcttctcttcggagttcttaacgctgctcatgaagaaacaacatccgaaacagtcacatcgcatcgcgttaaagaagaagaagttacctcagaatccgaggttattgaagcaataccggcgaaggagaagtcgtaccgaggtgttaggaggcgtccatggggtaaattcgcagcggagataagagattctacgagaaatggggttcgagtatggttagggacatttgatagcgcggaagatgctgctttagcttatgatcaagctgcgttttcaatgaggggtaattctgccattttgaattttccagtggagaccgttagggattcgctacgtgacatgaaatgtcacgtagacgatgattgctcccctgtagtggcgcttaaaaagcgccactccatgaggaaaaggagcacgaattccaaaaaagttaatagtattagtaaagtagtgagggaagttaaaatggaaaatgtaaataatgtagttgtgtttgaagatttgggtgctgattatttagaacaacttttaagtagtagttcaagtgatcaaagtagttgtgatgcaacttattttagtccatggtaa

SEQ ID NO: 52 - DNA, 486 bp, Solanum lysopersicum
atggttccaactcctcaaagtgatttacctcttaatgagaatgactcacaagagatggtattatatgaagttcttaatgaagctaatgctctaaatattccttatttaccccaacgaaatcaattactccctagaaataatattcttcgtccattacagtgcataggcaagaaatacagaggagtacgacgtcgtccgtgggggaaatacgctgcggaaattcgcgattcggctagacatggtgcgagagtatggctaggtacgttcgaaactgctgaagaagctgcgttagcttatgatagagcggcttttagaatgcgaggtgctaaggcactacttaattttccatctgaaatagtgaacgcctctgtttcagtagacaaattaagtttgtgctcaaatagttacactacgaataataattcagattcaagtttaaatgaagtttcaagtggaactaatgatgtatttgaatcaagatgttaa

SEQ ID NO: 53 - DNA, 1119 bp, Solanum lysopersicum
atgtgtggtggtgcaattatctccgatttggtacctcctagccggatttctcgccggttaaccgctgattttctatggggtacatccgatctgaacaagaagaagaagaaccctagtaattaccactcaaagcccttgaggtctaagtttattgaccttgaagatgaatttgaagctgactttcagcacttcaaggataattctgatgatgatgatgatgtgaaggcatttggccccaaatccgtgagatctggtgattcaaactgcgaagctgacagatcctccaagagaaagaggaagaatcagtaccgggggatcagacagcgtccttggggtaagtgggcagctgaaatacgtgatccaaggaaaggtattcgagtctggcttggtactttcaattcagccgaagaggcagccagagcttatgatgctgaggcgcgaaggatcagaggcaagaaagctaaggtgaactttcctgatgaagctccagtgtctgtttcaagacgtgctattaagcaaaatccccaaaaggcacttcgtgaggaaaccctgaacacagttcagcccaacatgacttatattagtaacttggatggtggatctgatgattcgttcagttttttcgaagagaaaccagcaaccaagcagtacggcttcgagaatgtgtcttttactgctgtagatatgggactgggctcagtttccccttcagctggtacaaatgtttacttcagctctgatgaagcaagtaacacttttgactgctctgatttcggttgggctgaaccgtgtgcaaggactccagagatctcatctgttctgtcggaagttctggaaaccaatgagactcattttgatgatgattccagaccagagaaaaaactgaagtcctgttccagcacttcattgacagttgacggtaacactgtgaacacgctatctgaagagctatcggcttttgaatcccagatgaagttcttgcagatcccatatctcgagggaaattgggatgcatcggttgatgccttcctcaatacaagtgcaattcaggatggtggaaacgccatggacctttggtccttcgatgatgtaccttctttaatgggaggtgcctactaa

SEQ ID NO: 54 - DNA, 984 bp, Solanum lysopersicum
atgtgtggtggttctataatctccgattacatagaccctagccggacttctcgccggctcaccgccgagtttctatggggtcgtttcgatctcggtaagaagcaaaaaaatcccaacaattatcactctaaagctaagcatttgcgatctgaagttgttgacgactttgaagccgattttcaggacttcaaagagttatccgatgatgaggatgttcaagtcgatgtcaagccatttgccttctctgcttccaaacactctactggttccaaatctttgaaaactgttgattcagacaaggatgctgctgctgataaatcctctaagagaaagaggaagaatcaatatagagggatcagacagagaccttggggtaagtgggcagctgaaatacgtgacccaaggaaaggggttcgggtctggctgggaaccttcaatactgcagaagaagctgccaaagcttatgatattgaggcgaggaggatcagaggcaagaaggctaaggtaaactttcctgatgaagctcccgcccctgcatcaagacacactgttaaggtgaatcctcagaaggtccttcctgaggagagcctgtattcacttcagtccgactcagcaatcatgaacagcgtggaggatgaccattatgattcttttggattttttgaagagaaacccatgacaaaacagtatggatatgagaatgggagcagtgcttctgcagatacgggatttggttcgttcgtcccttcagctggcggtgatatctacttcaactctgatgtaggaagcaactcttttgaatgctctgattttggttggggagagccatgctccaggactccagagatatcatctgttctgtcagctgctattgaatgtaatgaagctcaatttgttgaagatgccaattctcagaaaaagttgaaatcatgcaccaacaaccccgtagctgatgatggaaacccccgttactatggtacctga

SEQ ID NO: 55 - DNA, 747 bp, Solanum lysopersicum
atgacggaaaattcagttccggtgattaaattcactcaacacatagtaactacaaacaagcatgttttttctgagcataacgaaaaatccaattcagagttacaaagagttgtgaggattatacttacagatgccgatgctacagattcttccgatgatgaaggccggaatactgtacggagagtgaagaggcacgtgacggagatcaaccttatgccgtcaaccaaatcgatcggcgacagaaaacgaagatcggtgtctccggattctgacgtcactcgtcggaaaaagtttagaggcgttcgtcaaagaccgtggggtcgttgggctgcagagattcgggacccgaccgggggaaaacgggtgtggttgggtacttatgacaccccagaagaagcagctgtcgtttacgataaagctgcagttaagctcaaaggtcctgacgccgttaccaattttccggtatcaacaacggcggaggtaacggtgacggttacggaaaccgaaaccgagtctgttgccgacggtggagataaaagcgaaaacgatgtcgctttgtcacccacctcagttctctgtgacaatgattttgcgccgtttgacaatctagggttctgcgaagtggatgcttttggtttcgacgttgattcacttttccggctgccggattttgctatgacggagaaatactacggcgatgaattcggcgaatttgactttgacgattttgcccttgaagctcgatag

SEQ ID NO: 56 - DNA, 705 bp, Solanum lysopersicum
atgtattcaaattgtgaactagaaaatgatttttcagtactcgaatcaattagaagatacttacttgaagattgggaagctccattaacgagctctgaaaactcaacatcctcagagttcagccggagcaacagcattgaatccaatatgtttagtaattcatttgattatacacctgaaatttttcaaaatgatattcttaatgaaggatttggatttggatttgaattcgagacttctgattttataatccctaaattagagtcacaaatgtcaatcgaatcacctgaaatgtggaatttaccggaatttgtggctccattagagacggcggcggaggtgaaagttgaaacaccggttgagatgacaactacgacgacgaagccaaaggcaaagcattatagaggtgtgagagtgaggccatgggggaaattcgcggcggaaattagagatccggcgaaaaatggagcacgagtttggctcggtacatatgagacggcggaggatgcggcgttggcttacgacaaggcggcttttcgcatgcggggatcacgtgcattgctgaattttccgttgaggattaattccggtgaaccggatcctgttagagttggatcgaagagatcgtcaatgtcgccggagcattgttcatcggcgtcgtcgacgaagaggaggaagaaggttgctcgtggaacaaagcaataa

(19) United States
(12) Patent Application Publication
(10) Pub. No.: US 2011/0165678 A1
(43) Pub. Date: Jul. 7, 2011

(54) New Tomato Ethylene Response Factors and Uses Thereof

(75) Inventors: Mondher Bouzayen, Auzeville-Tolosane (FR); Alain Latche, Toulouse (FR); Jean-claude Pech, Toulouse (FR); Julien Pirrello, Toulouse (FR); Farid Regad, Toulouse (FR)

(73) Assignee: INSTITUT NATIONAL POLYTECHNIQUE DE TOULOUSE, Toulouse (FR)

(21) Appl. No.: 13/062,122

(22) PCT Filed: Sep. 4, 2009
(86) PCT No.: PCT/EP2009/061470

§ 371 Date: Mar. 3, 2011

(30) Foreign Application Priority Data
Sep. 4, 2008 (EP) 08163692.0

(51) Int. Cl. C12N 5/10; C07K 14/415; A01H 1/00; A01H 5/00; A01H 5/10

(52) U.S. Cl. 435/419; 530/370; 800/278; 800/298; 800/317.4

(57) ABSTRACT

The present invention concerns a tomato transcription factor of the Ethylene Responsive Factor family (ERF), having an amino acid sequence selected among the group consisting in:
a protein having an amino acid sequence as shown in one of SEQ ID NO 1 to NO 28,
a variant thereof,
a functional fragment thereof and
   a functional homologous sequence thereof.

The plant hormone ethylene is involved in the control of a myriad of plant developmental processes and is also known to play an active role in plant responses to biotic and abiotic stresses. It is well established that this plant hormone exerts its effects by modulating the expression of a large number of genes through the activation of specific members of the Ethylene Response Factor (ERF) gene family. ERFs are plant specific transcriptional regulators encoded by one of the largest gene family known in plant kingdom. Because ERFs are down-stream mediators of ethylene action, they are the main actors for the amplification and diversification of plant responses to ethylene.

The making of a fruit is a developmental process unique to plants, involving a complex network of interacting genes and signalling pathways. In the case of fleshy fruit, this process involves three main stages: (a) fruit set, (b) fruit development, and (c) fruit ripening. This latter stage is crucial for fruit quality and most of the sensory and health promoting compounds accumulate during the ripening step.

Ripening is a genetically programmed process orchestrated by complex interplay between endogenous hormones and environmental cues. The regulated changes at the level of gene expression are the first steps leading to the metabolic changes associated with fruit ripening. In climacteric fruit type like tomato, the phytohormone ethylene is a key regulator of this process.

However, while molecular and genetic evidences are becoming available showing that ethylene triggers and co-ordinates fruit ripening, the developmental cues required to signal a readiness to ripen remain unknown and the mechanism by which the fruit developmental program switches into a ripening process is yet to be elucidated.

Transcription factors from the ERF type are thought to be, at least partly, responsible of the complex network of metabolic activations associated with the ripening process, nevertheless, the mechanism by which they select ripening-specific genes remains largely unknown.

### PRIOR ART

At first, Ethylene Responsive Factors were isolated as GCC box binding proteins from tobacco (Ohme-Takagi and Shinshi, 1995). Later, ERFs have been identified in numerous plants including Arabidopsis Thaliana and Solanum lycopersicum (tomato).

A highly conserved DNA binding domain, known as the “AP2/ERF domain”, consisting of 58 to 59 amino acids, is the unique structural feature of common to all factors belonging to this protein family. This ERF domain binds to DNA as a monomer, with high affinity.

ERFs can act as either transcriptional activators or repressors for GCC box-dependant gene expression (Fujimoto et al., Plant Cell, 2000).

ERFs have been shown to be involved in normal and abnormal plant processes such as plant defense, osmotic stress tolerance, and seed germination.

In tomato, new members of the ERF family were identified in 2003 and their respective role and expression pattern were studied (Tournier et al., FEBS Letters, 2003). Four distinct classes of ERF were described, based on functionalities and structures of each subtype.

Later, the physiological function of one of the identified ERF was extensively studied. In particular, overexpression of said ERF gene in transgenic tomato line results in premature seed germination and increased ethylene sensitivity. Interestingly, in such transformed plants, the mannanase2 encoding gene is upregulated in seeds (Pirrello et al., 2006).

This preliminary data indicate that each ERF controls a specific subset of ethylene-regulated genes. Therefore, identifying a set of ripening-associated genes that are under the regulation of a given ERF will open new prospects towards the targeted control of genes involved in specific metabolic & developmental pathways, and more particularly in ripening control.

### DESCRIPTION OF THE INVENTION

The present invention is related to new ERF proteins, identified in tomato, classified according to their structural characteristics, expression pattern and physiological functions.

The invention is also related to the modulation of the expression of genes encoding said ERFs, these activation or repression being used to obtain new interesting phenotypes of tomato plants.

Moreover, mapping of genes encoding ERF proteins, and identification of genetic markers QTL linked to these genes, are claimed here.

Screening of various cDNA libraries from different plant tissues and organs allowed the isolation of complete cDNA clones for 28 tomato ERFs. Complete amino acid sequences of the ERF proteins are provided in FIG. 1 and in the Sequence Listing; corresponding polynucleotide sequences of the cDNA are provided in the sequence listing.

In silico analysis allowed to classify the encoded proteins based on their structural features and by comparison with the corresponding Arabidopsis (Arabidopsis thaliana) and rice (Oryza sativa L. subsp. japonica) orthologs. Phylogenetic analyses defined eight ERF sub-classes in the tomato; each of them display distinct specific features (FIGS. 2 and 3).

The spatio-temporal pattern of expression of some members of the tomato ERF gene family was established which indicated that some ERFs are preferentially expressed in the fruit, other ERFs in flower organ and a few in vegetative tissues, while the remaining ERFs displays a constitutive pattern of expression (FIG. 4).

The ability of each ERF to regulate in vivo the activity of ethylene-responsive promoters, containing or not a GCC box, was assayed. Depending on the sub-class to which they belong, ERF proteins can act either as positive or negative regulators of GCC-containing promoters.

Moreover, some ERFs can also modulate the transcriptional activity of native ethylene-responsive promoters lacking the canonical GCC box (FIG. 5).

All together, the structural diversity, the preferential binding to target promoters as well as the differential patterns of expression, provide new insights on how ERF proteins mediate ethylene responses in highly selective and specific manner.

Among the numerous applications that arise from the isolation and functional characterization of tomato ERFs, the following are of first importance:
Obtaining ERF-drived markers for plant breeding via marker-assisted classical genetics approaches;
Selection of naturally occurring or chemically induced mutants having commercially relevant phenotypes including fruit traits, tolerance to biotic and abiotic stresses;
Up-regulating and down-regulating the expression of ERFs via transgenesis in homologous or heterologous hosts to generate novel and advantageous plant properties.

### DETAILED DESCRIPTION OF THE INVENTION

The present invention is related to new transcription factors from tomato, belonging to the Ethylene Responsive Factor family (ERF), showing an amino acid sequence chosen among SEQ ID NO 1 to NO 28, a variant thereof, a functional fragment thereof and a functional homologous sequence thereof.

The term of <<transcription factor>> designates a protein that binds to promoters of genes, using DNA binding domains, and controls the transcription of the gene, i.e. the level of expression of the gene.

The term of “Ethylene Responsive Factor” or “ERF” designates a transcription factor having as DNA binding domain, a domain called “AP2/ERF” consisting of 58 to 59 amino acids, highly conserved among all the factors of the family.

The term “functional fragment” means according to the invention that the sequence of the polypeptide may include less amino-acid than shown in SEQ ID N° 1 to N° 28 but still enough amino acids to confer the Ethylene Responsive Factor activity. It is well known in the art that a polypeptide can be modified by substitution, insertion, deletion and/or addition of one or more amino acids while retaining its DNA binding activity. For example, substitutions of one amino-acid at a given position by a chemically equivalent amino-acid that do not affect the functional properties of a protein are common. For the purposes of the present invention, substitutions are defined as exchanges within one of the following groups:
Small aliphatic, non-polar or slightly polar residues: Ala, Ser, Thr, Pro, Gly
Polar, negatively charged residues and their amides: Asp, Asn, Glu, Gln
Polar, positively charged residues: His, Arg, Lys
Large aliphatic, non-polar residues: Met, Leu, Ile, Val, Cys
Large aromatic residues: Phe, Tyr, Trp.

Thus, changes which result in substitution of one negatively charged residue for another (such as glutamic acid for aspartic acid) or one positively charged residue for another (such as lysine for arginine) can be expected to produce a functionally equivalent product.

The positions where the amino acids are modified and the number of amino acids subject to modification in the amino acid sequence are not particularly limited. The man skilled in the art is able to recognize the modifications that can be introduced without affecting the activity of the protein. For example, modifications in the N- or C-terminal portion of a protein would not be expected to alter the activity of a protein.

The term “functional homologous” and “variant” refers to polypeptides submitted to modifications such as defined above while still retaining the original enzymatic activity.

In a specific embodiment of the invention, the polypeptide of the present invention have at least 70% identity with the sequences shown as SEQ ID N° 1 to SEQ ID N° 28, preferentially at least 80% identity and more preferentially at least 90% identity.

Methods for determination of the percentage of identity between two protein sequences are known from the man skilled in the art. For example, it can be made after alignment of the sequences by using the software CLUSTALW available on the website http://www.ebi.n.uk/clustalw/ with the default parameters indicated on the website. From the alignment, calculation of the percentage of identity can be made easily by recording the number of identical residues at the same position compared to the total number of residues. Alternatively, automatic calculation can be made by using for example the BLAST programs available on the website http://www.ncbi.nlm.nih.gov/BLAST/ with the default parameters indicated on the website.

In a particular embodiment of the invention, ERFS are selected among the proteins having an amino acid sequence as shown in SEQ ID NO 1 to 28, and more preferentially in SEQ ID NO 1 to 6.

In another embodiment of the invention, ERFS are selected among DNA fragments having a nucleotidic sequence as shown in one of SEQ ID NO 29 to NO 56 or a functional homologous sequence thereof. SEQ ID NO 29 encodes for a protein having the amino acid sequence as shown in SEQ ID NO 1, SEQ ID NO 30 encodes for a protein having the amino acid sequence as shown in SEQ ID NO 2, and so on.

According to the invention, the tomato ERFs present specific features, and in particular at least one of the following structural features:
a nuclear localization signal; almost all ERFs according to the invention show this signal, except ERF.D1, B.1 and H1. Presence of a nuclear localization signal strongly supports that the encoded ERF proteins are targeted to the cell nucleus, and therefore act, positively or negatively, on transcription regulation;
an acidic domain; almost all ERF according to the invention show an acidic domain, except ERF.A1, D1, D2, D3 and F4. Presence of an acidic domain suggests that the corresponding ERF proteins display transcriptional activation properties;
an EAR domain; presence of a putative EAR motif strongly supports that the corresponding ERF proteins display some transcriptional repression properties. This characteristic is specific to ERF belonging to the class F:F1 to F5;
a putative MAP kinase phosphorylation site; presence of a MAP kinase phosphorylation site suggests that the corresponding ERF proteins bind efficiently on the GCC box present in DNA;
a sequence “MCGGAII/L”; this structural characteristic is found in all factors of class E. Its function is still unknown;
a sequence CMX-1; this structural characteristic is found in all factors of class D but D4. Its function is still unknown;
a glutamine rich region; this structural characteristic is found in factors ERF.D3 and D4. Its putative function is an activation of the transcription. Some ERFs according to the invention present a combination of two or more of the features presented above.

The spatio-temporal pattern of expression of twenty-four members of the tomato ERF gene family was established, which indicated that some ERFs are preferentially expressed in the fruit, others in flower organ or in both flower and fruit, a few are expressed mainly in vegetative tissues, while the remaining ERFs display a constitutive pattern of expression.

The knowledge of the expression pattern of these ERFs is of primary importance to anticipate the physiological effects of a modulation of expression of these factors.

The invention is in particular related to an ERF involved in the regulation of the sugar level in the fruit, wherein the ERF has an amino acid sequence as shown in SEQ ID NO 1 or in SEQ ID NO 2, or a variant thereof, or a functional fragment thereof or a functional homologous sequence thereof. As shown in FIG. 11, the over-expression of a dominant negative (repressor) form of ERF.B.3 (SEQ ID NO 1) and ERF.F.5 (SEQ ID NO 2) lead to significative modifications of the sugar level in the fruits of the transgenic lines.

ERF.B.3 (SEQ ID NO 1) has a constitutive expression in the plant, and is a transcriptional activator of responsive genes Inhibiting its activity leads to a decrease of sugar content in the fruit. It is probable that overexpression of this ERF could induce an increase of the sugar content in fruits. FIG. 6 also shows that this ERF is involved in the regulation of the size of the fruit.

ERF.F.5 (SEQ ID NO 2) is mainly expressed in the fruit. It is a transcriptional inhibitor of responsive genes. Increasing of its transcriptional activity leads to an increase of sugar content in the fruit. It is probable that overexpression of this ERF could induce an increase of the sugar content in fruits

Interestingly, these transcriptional factors ERF.B.3 (SEQ ID NO 1) and ERF.F.5 (SEQ ID NO 2) are able to regulate the transcriptional activity of ethylene-responsive promoters lacking the canonical GCC box (see FIG. 5).

Another aspect of the invention concerns an ERF involved in the regulation of the shininess of the fruit, wherein the ERF has an amino acid sequence as shown in SEQ ID NO 3 or a variant thereof, or a functional fragment thereof or a functional homologous sequence thereof. As shown in FIG. 10, the over-expression of a dominant negative (repressor) form of ERF.H.1 (SEQ ID NO 3) lead to a significative improvement of the shininess of the fruit.

Other ERFs according to the invention are slightly involved in the shininess of the fruit: ERF.C.3 (SEQ ID NO 5) and ERF.E.1 (SEQ ID NO 4), as shown in FIG. 10, have an effect on the shininess.

Another aspect of the invention concerns an ERF involved in the regulation of the size of the fruit, wherein the ERF has an amino acid sequence as shown in SEQ ID NO 4 or a variant thereof, or a functional fragment thereof or a functional homologous sequence thereof. As shown in FIG. 8, the expression of repressor of ERF.E.1 (SEQ ID NO 4) lead to fruit significantly lighter than wild-type fruits.

Another aspect of the invention concerns an ERF involved in the regulation of the shape of the fruit, wherein the ERF has an amino acid sequence as shown in SEQ ID NO 5 or a variant thereof, or a functional fragment thereof or a functional homologous sequence thereof. As shown in FIG. 7, the over-expression of a dominant negative (repressor) form of ERF.C.3 (SEQ ID NO 5) lead to fruit presenting an elongated shape, in comparison with the wild-type fruit.

Another aspect of the invention concerns an ERF involved in the regulation of the color of the fruit, wherein the ERF has an amino acid sequence as shown in SEQ ID NO 4 or in SEQ ID NO. 5, or a variant thereof, or a functional fragment thereof or a functional homologous sequence thereof. As shown in FIG. 9, the over-expression of a dominant negative (repressors) form of ERF.C.3 (SEQ ID NO 5) and ERF.E.1 (SEQ ID NO 4) lead to a difference in the color, these fruits being less red than the wild-type fruits. It is therefore probable that overexpression of these proteins in transgenic tomato lines would lead to the obtention of reder fruits.

Another aspect of the invention concerns an ERF involved in the regulation of the production & response of fruits to ethylene, wherein the ERF has an amino acid sequence as shown in SEQ ID NO 6 or a variant thereof, or a functional fragment thereof or a functional homologous sequence thereof.

Each of the tomato ERFs according to the invention can act positively or negatively on the transcription of responsive genes. Indeed, the ERF proteins are known to modulate transcription of ethylene-responsive genes via binding to the so-called GCC box, a cis-elements found in the promoter region of ethylene-regulated genes.

A “responsive gene” is defined as a gene having a promoter on which the ERF can bind, and the expression level of said gene is modulated positively or negatively by the binding of the ERF.

Each ERF has the functional ability to regulate in vivo the activity of ethylene-responsive promoters. This ability was assayed by using a dedicated transient expression system.

The data presented in example 2 (below) indicate that depending on the sub-class to which they belong, ERF proteins can act either as positive or negative regulators of GCC-containing promoters.

Surprisingly, inventors show also that some ERFs can also modulate the transcriptional activity of native ethylene-responsive promoters lacking the canonical GCC box (FIG. 5 and table 2).

In a specific embodiment of the invention, tomato ERFs according to the invention are proteins encoded by a gene localized on the genome close to at least one genetic marker such as a QTL (Quantitative Trait Locus). A quantitative trait locus is a region of DNA that is associated with a particular phenotypic trait, since they are closely linked to the genes that underlie the trait in question. QTLs can be molecularly identified (for example, with PCR or AFLP) to help map regions of the genome that contain genes involved in specifying a quantitative trait.

The man skilled in the art knows how to realize the genetic mapping of the ERFs and associated QTLs on the tomato genome. This will allow the implementation of association genetics strategy in order to uncover putative co-localization of a particular ERF with fruit quality QTLs and therefore to identify candidate ERF genes that are key regulator of specific aspects of fruit ripening and quality.

The invention is also related to a plant cell and to plants having a different expression of at least one of the ERF according to the invention. This difference may be an increased or a decreased expression level of the gene, an increased or a decreased expression level of the protein, or an increased or a decreased activity of the protein.

Such modulation of expression of genes or proteins are well known by the man skilled in the art, and can be obtained by various means including but not limited to genetic manipulation, deletion of a gene, replacement of a native promoter with a stronger one, stabilization or destabilization of the encoded RNA messenger, stabilization, degradation or exportation of the encoded protein.

The modulation of the activity of a protein can also be achieved by various means known by the man skilled in the art, including but not limited to introduction of point mutations into the gene leading to the translation of a protein with a different level of activity than the native protein.

This difference of expression may be observed in all cells of the plant, or only in part of them. This different level of expression may be constitutive or induced at a specific time, by addition of inducers.

The plant cell according to the invention may have in particular a genetic alteration in the regulatory or in the coding sequence of at least one ERF according to the invention.

The genetic alteration may result of a selection process of naturally occurring mutations, or of a genetic manipulation of the plant.

The genetic alteration may be a mutation, a deletion or an overexpression of the gene encoding the ERF according to the invention.

Genetic alterations according to the invention may result in different phenotypes, depending on the ERF that is mutated, deleted or overexpressed.

In an embodiment of the invention, at least one ERF encoding gene has its expression enhanced. To obtain an increase of the expression of a gene encoding a specific ERF, the man skilled in the art knows different methods, and for example:
Replacement of the endogenous promoter of said gene encoding an ERF with a stronger promoter;
Introduction into the cell of an expression vector carrying said gene encoding a specific ERF;
Introducing additional copies of said gene encoding a specific ERF into the chromosome.

In another embodiment of the invention, at least one ERF encoding gene has its expression inhibited. To obtain the attenuation of the expression of a gene, different methods exist, and for example:
Introduction of a mutation into the gene, decreasing the expression level of this gene, or the level of activity of the encoded protein;
Replacement of the natural promoter of the gene by a low strength promoter, resulting in a lower expression;
Use of elements destabilizing the corresponding messenger RNA or the protein;
Deletion of the gene if no expression is needed.

In another embodiment of the invention, there is a partial or total inhibition of the transcriptional activity of at least one ERF in the plant cell. To obtain this inhibition of activity, a classical method is the expression of the dominant negative (repressor) form of said ERF, inducing the suppression of the expression of the target genes. Construction of dominant negative forms are fully explained in (Hiratsu et al., 2003) and in example 4 below.

Genetic transformation of plants are now well known in the art, comprising introducing a new gene fragment in a plant cell and then regenerating a plant form the cell. The new gene fragment is preferably introduced with known techniques of particle bombardment and/or infection with a transformed Agrobacterium. Regeneration procedure are also well known in the art and documented for numerous plant today. The new gene fragment is preferably integrated into the plant cell genome. New techniques of homologous recombination and gene replacement are also known today to be effective in plant cells.

The term <<transformation>> refers to the incorporation of exogenous nucleic acid by a cell, this acquisition of new genes being transitory (if the vector carrying genes is cured) or permanent (in the case the exogenous DNA is integrated chromosomally).

The term <<vector>> refers to an extra-chromosomal element carrying genes or cassettes, that is usually in the form of a circular double-stranded DNA molecules, but may be a single strand DNA molecule, too. Both terms “vector” and “plasmid” are used indifferently.

The invention is also related to a plant containing at least one plant cell such as described above.

The term “plant” according to the invention means any plant, monocotyledonous (small leafs) or dicotyledonous (broad leafs) and particularly crops having a commercial interest in the agricultural industry, more particularly crops selected among the group consisting of acacia, alfalfa, aneth, apple, apricot, artichoke, arugula, asparagus, avocado, banana, barley, beans, beet, blackberry, blueberry, broccoli, brussels sprouts, cabbage, canola, cantaloupe, carrot, cassaya, cauliflower, celery, cherry, cilantro, citrus, clementine, coffee, corn, cotton, cucumber, Douglas fir, eggplant, endive, escarole, eucalyptus, fennel, figs, forest trees, gourd, grape, grapefruit, honey dew, jicama, kiwifruit, lettuce, leeks, lemon, lime, loblolly pine, mango, melon, mushroom, nut, oat, okra, onion, orange, an ornamental plant, papaya, parsley, pea, peach, peanut, pear, pepper, persimmon, pine, pineapple, plantain, plum, pomegranate, poplar, potato, pumpkin, quince, radiata pine, radicchio, radish, rapeseed, raspberry, rice, rye, sorghum, Southern pine, soybean, spinach, squash, strawberry, sugarbeet, sugarcane, sunflower, sweet potato, sweetgum, tangerine, tea, tobacco, tomato, turf, a vine, watermelon, wheat, yams, and zucchini.

In a preferred embodiment, said plants are selected among fruiting and flowering vegetables, including Armenian cucumber (Cucumis melo Flexuosus group), Eggplant (Solanum melongena), Avocado (Persea americana), Bell pepper (Capsicum annuum), Bitter melon (Momordica charantia), Caigua (Cyclanthera pedata), Cape Gooseberry (Physalis peruviana), Cayenne pepper (Capsicum frutescens), Chayote (Sechium edule), Chili pepper (Capsicum annuum Longum group), Cucumber (Cucumis sativus), Globe Artichoke (Cynara scolymus), Luffa (Luffa acutangula, Luffa aegyptiaca), Malabar gourd (Cucurbita ficifolia), Parwal (Trichosanthes dioica), Perennial cucumber (Coccinia grandis), Pumpkin (Cucurbita maxima, Cucurbita pepo), Pattypan squash Snake gourd (Trichosanthes cucumerina), Squash (Cucurbita pepo), Sweetcorn (Zea mays), Sweet pepper (Capsicum annuum Grossum group), Tinda (Praecitrullus fistulosus), Tomato (Solanum lycopersicum), Tomatillo (Physalis philadelphica), Winter melon (Benincasa hispida), West Indian gherkin (Cucumis anguria), Zucchini (Cucurbita pepo), and more preferably tomato plants.

Preferred phenotypes of plants and particularly of vegetable plants, more particularly of tomato plants according to the invention, are presented below.

In a specific embodiment of the invention, a plant having a genetic alteration in one of the gene encoding for an ERF according to the invention shows an increased tolerance to viral, bacterial and/or a fungal infections.

An increased plant tolerance to viral infections may be in particular an increased tolerance to one of the following virus: Tomato mosaic virus (ToMV), Tomato spotted wilt virus (TSWV), Tobacco and tomato ringspot virus, Curly top virus (Curly top virus), Potato virus Y virus, Tomato bushy stunt virus, Tomato etch virus, Tomato fern leaf virus (TYLCV), Tomato mottle virus, Tomato necrosis virus, Tomato spotted wilt virus, Tomato yellow leaf curl virus, Tomato yellow top virus, Tomato bunchy top virus, Tomato planto macho virus, Aster yellows virus, Tomato big bud virus, Torrado virus, Marchito Virus.

The man skilled in the art knows how to determine if a plant is resistant or not to a specific virus, bacteria or fungus using screening method well known to the one skilled in the art of agronomy, agrochemistry and agriculture.

An increased plant tolerance to bacterial infections may be in particular an increased tolerance to one of the following bacterial infections: Bacterial canker (Clavibacter michiganensis subsp. Michiganensis), Bacterial speck (Pseudomonas syringae pv. Tomato), Bacterial spot (Xanthomonas campestris pv. Vesicatoria), Bacterial stem rot and fruit rot (Erwinia carotovora subsp. Carotovora), Bacterial wilt (Ralstonia solanacearum), Pith necrosis (Pseudomonas corrugata), Syringae leaf spot (Pseudomonas syringae pv. Syringae).

An increased plant tolerance to fungal infections may be in particular an increased tolerance to one of the following fungal infections: Alternaria stem canker (Alternaria alternata fsp. lycopersici), Anthracnose (Colletotrichum coccodes, Colletotrichum dematium, Colletotrichum gloeosporioides, Glomerella cingulata), Black mold rot (Alternaria alternata, Stemphylium botryosum, Pleospora tarda, Stemphylium herbarum, Pleospora herbarum, Pleospora lycopersici, Ulocladium consortiale, Stemphylium consortiale), Black root rot (Thielaviopsis basicola, Chalara elegans), Black shoulder (Alternaria alternata), Buckeye fruit and root rot (Phytophthora capsici, Phytophthora drechsleri, Phytophthora parasitica), Cercospora leaf mold (Cercospora fuligena), Charcoal rot (Macrophomina phaseolina), Corky root rot (Pyrenochaeta lycopersici), Didymella stem rot (Didymella lycopersici), Early blight (Alternaria solani), Fusarium crown and root rot (Fusarium oxysporum f.sp. radicis-lycopersici), Fusarium wilt (Fusarium oxysporum f.sp. lycopersici), Gray leaf spot (Stemphylium botryosum fsp. lycopersici, Stemphylium lycopersici, Stemphylium floridanum, Stemphylium solani), Gray Mold (Botrytis cinerea, Botryotinia fuckeliana), Late blight (Phytophthora infestans), Leaf mold (Fulvia fulva), Phoma rot (Phoma destructiva), Powdery mildew (Oidiopsis sicula, Leveillula taurica), Pythium damping-off and fruit rot (Pythium aphanidermatum, Pythium arrhenomanes, Pythium debaryanum, Pythium myriotylum, Pythium ultimum), Rhizoctonia damping-off and fruit rot (Rhizoctonia solani, Thanatephorus cucumeris), Rhizopus rot (Rhizopus stolonifer), Septoria leaf spot (Septoria lycopersici), Sour rot (Geotrichum candidum, Galactomyces geotrichum, Geotrichum klebahnii), Southern blight (Sclerotium rolfsii, Athelia rolfsii), Target spot (Corynespora cassiicola), Verticillium wilt (Verticillium albo-atrum, Verticillium dahliae), White mold (Sclerotinia sclerotiorum, Sclerotinia minor).

In another embodiment of the invention, a plant, particularly a vegetable plant, more particularly a tomato plant having a genetic alteration in at least one of the gene encoding for an ERF according to the invention, shows an increased tolerance to insects attacks and/or increased tolerance to nematodes.

The man skilled in the art knows how to determine the resistance of a plant to a specific insect or nematode using screening method well known to the one skilled in the art of agronomy, agrochemistry and agriculture.

Preferentially, the genetic alteration in the regulatory or coding sequence of a member of the ERF family leads to increased plant tolerance to insects attacks such as: acarians, aphids, white flies, trips, caterpillar, leaf miner.

Preferentially, the genetic alteration in the regulatory or coding sequence of a member of the ERF family leads to increased plant tolerance to Nematodes such as: Root-knot (Meloidogyne spp.), Sting (Belonolaimus longicaudatus), Stubby-root (Paratrichodorus spp., Trichodorus spp.).

In another embodiment of the invention, a plant, particularly vegetable plant, more particularly a tomato plant having a genetic alteration in at least one of the gene encoding for an ERF according to the invention, shows an increased tolerance to abiotic stresses.

Abiotic stresses are for example: chilling, freezing, drought, salinity of the growth substrate, presence of heavy metals, wind, dehydration, high CO2 content, acid soils, basic soils.

In another embodiment of the invention, a plant, particularly a vegetable plant, more particularly a tomato plant having a genetic alteration in at least one of the gene encoding for an ERF according to the invention, shows modifications and in particular improvement of fruit properties.

A genetic alteration in the regulatory or coding sequence of a member of the ERF family leading to modifications of fruit properties such as: colour, shape, shininess, size, weight, shelf-life, maturation, response to ethylene, production of ethylene, flesh texture, sugar content, vitamin content, anti-oxydant content, firmness, freshness, mineral content, absence of seeds (Seedless), absence of Jelly (all-flesh), aroma content, flavour, volatile compounds composition, organoleptic properties.

In another embodiment of the invention, a plant, particularly a vegetable plant, more particularly a tomato plant having a genetic alteration in at least one of the gene encoding for an ERF according to the invention, shows modifications of plant agronomic properties.

A genetic alteration in the regulatory or coding sequence of a member of the ERF family can lead to modifications of plant agronomic properties such as such as: plant vigour, precocity, inter-node distance, number of flowers, fertility of flowers, plant architecture, absence of axillary shoots, parthenocarpy, apomixes.

In another embodiment of the invention, a plant, particularly a vegetable plant, more particularly a tomato plant having a genetic alteration in at least one of the gene encoding for an ERF according to the invention, shows modifications of seed properties.

A genetic alteration in the regulatory or coding sequence of a member of the ERF family can lead to modifications of seed properties such as such as: germination, yield, weight, size, conservation.

The invention is also related to a plant having a genetic alteration in the regulatory or in the coding sequence of a gene encoding the ERF protein having an amino acid sequence as shown in SEQ ID NO 1 or SEQ ID NO 2, and wherein said plant shows modifications of the sugar content in the fruit.

The invention is also related to a plant having a genetic alteration in the regulatory or in the coding sequence of a gene encoding the ERF protein having an amino acid sequence as shown in SEQ ID NO 3, and wherein said plant shows modifications in the shininess of the fruit.

The invention is also related to a plant having a genetic alteration in the regulatory or in the coding sequence of a gene encoding the ERF protein having an amino acid sequence as shown in SEQ ID NO 4, and wherein said plant shows modifications of the fruit size.

The invention is also related to a plant having a genetic alteration in the regulatory or in the coding sequence of a gene encoding the ERF protein having an amino acid sequence as shown in SEQ ID NO 5, and wherein said plant shows modifications of the fruit shape.

The invention is also related to a plant having a genetic alteration in the regulatory or in the coding sequence of a gene encoding the ERF protein having an amino acid sequence as shown in SEQ ID NO 4 or in SEQ ID NO 5, and wherein said plant shows modifications of fruit color.

The invention is also related to a plant having a genetic alteration in the regulatory or in the coding sequence of a gene encoding the ERF protein having an amino acid sequence as shown in SEQ ID NO 6, and wherein said plant shows modifications of the fruit shape, size, shininess or color, or of sugar content in the fruit, and/or modification in the production and/or response to ethylene.

The present invention also concerns a seed of a plant according to the invention, or comprising at least one plant cell according to the invention.

The present invention also concerns a method for obtaining a plant having new phenotypic characteristics, comprising introducing into a plant cell a genetic alteration in the regulatory or in the coding sequence of at least one gene encoding an ERF protein as define above and below and regenerating at least one fertile plant comprising said cell.

This genetic alteration leads in particular to a modification of the level of expression of at least one of the ERF encoding genes.

Preferably, the plant being regenerated is further crossed with another plant and seeds are harvested, said crossing step being eventually repeated at least one time.

The invention also concerns seeds and progenies of plants of the invention, wherein said seeds and progenies have a genetic alteration in the regulatory or in the coding sequence of a gene encoding an ERF protein as defined above and below.

### DRAWINGS

FIG. 1: Complete amino acid sequences of the twenty-eight claimed tomato ERF proteins.

FIG. 2: Structural features of different subclasses of tomato ERF.

FIG. 3: Phylogenetic tree of Arabidopsis Thaliana and Solanum lycopersicum ERF proteins.
A—Homology of the amino acid sequence of the whole protein.
B—Homology of the amino acid sequence of the AP2/ERF domain only.

FIG. 4: Expression pattern of the ERF in different plant tissue and organs. Quantitative RT-PCR of ERF transcript in total RNA samples extracted from Stem (St), Roots (R), Leaves (L), Flower (Fl), Early Immature Green Fruit (EIMG), Mature Green Fruit (MG), Breaker Fruit (B), Breaker +2 days (B+2), Breaker +7 days (B+7).

FIG. 5: ERF activity on synthetic or native complex promoter assessed by transient expression in a single cell system. The fluorescence of a reporter gene was assessed by flux cytometrie.

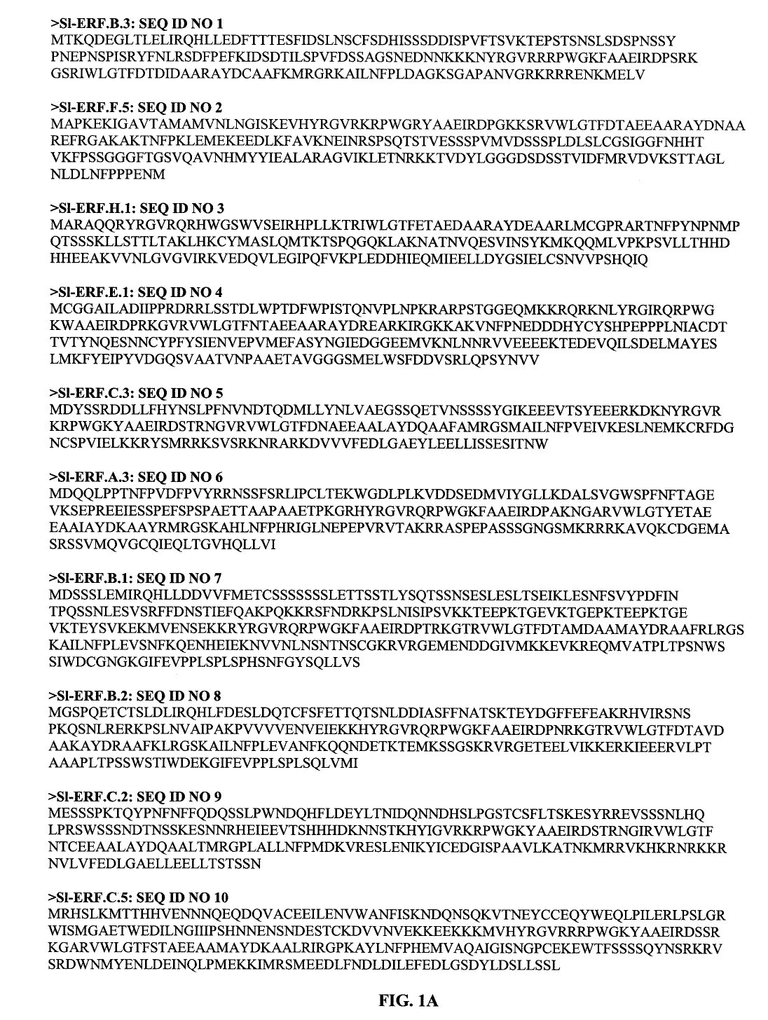

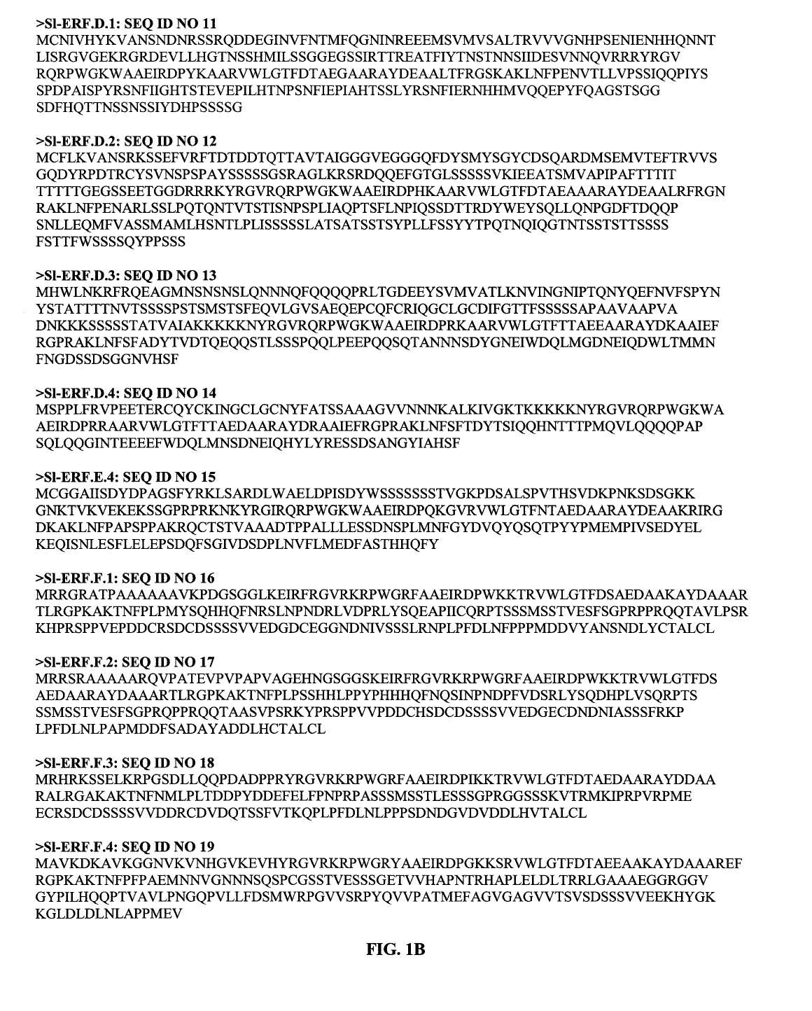

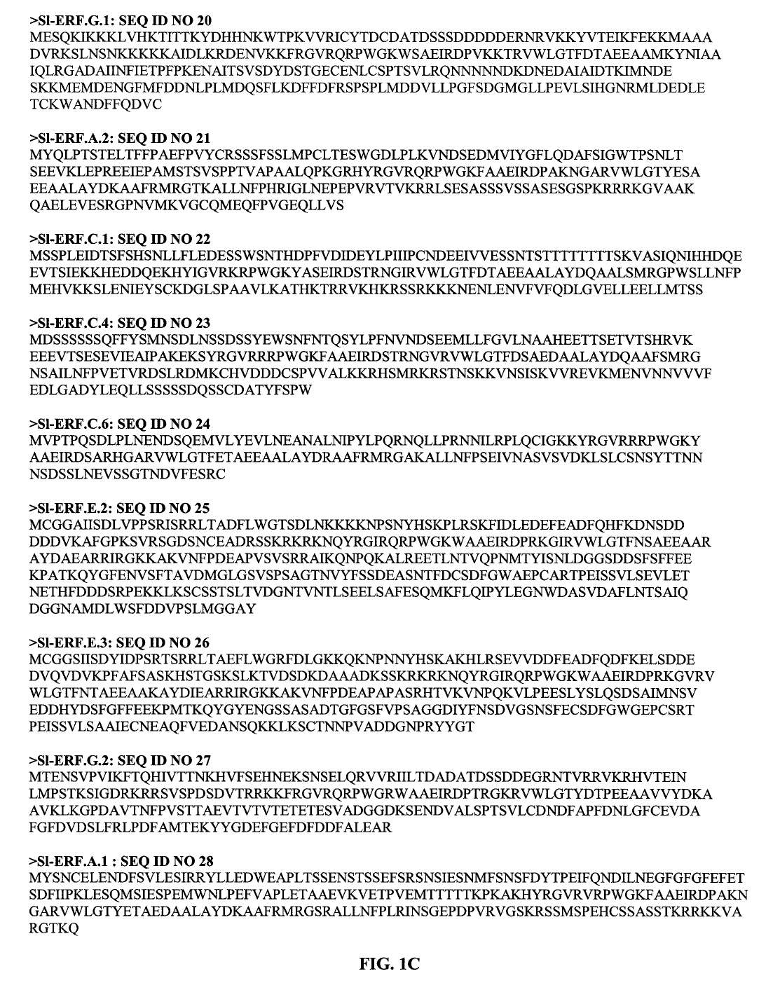

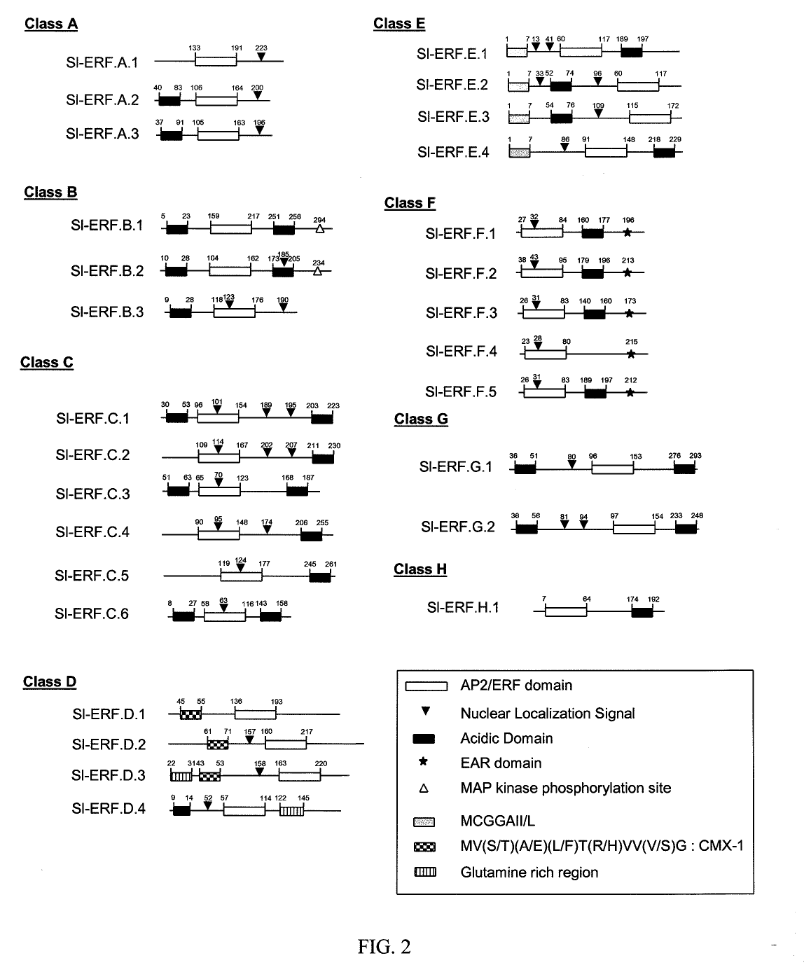

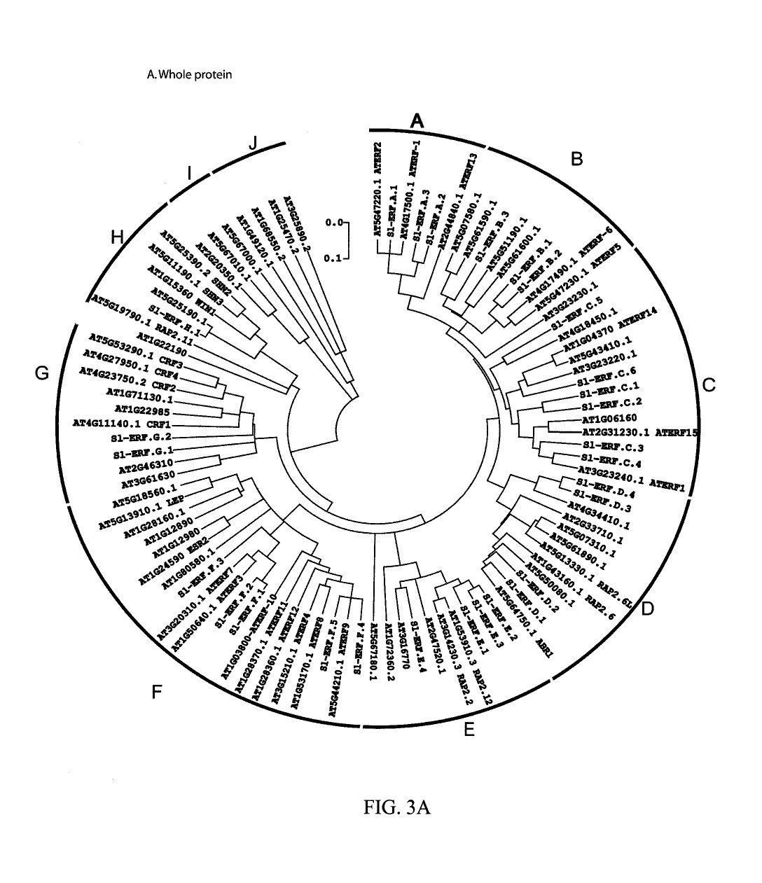

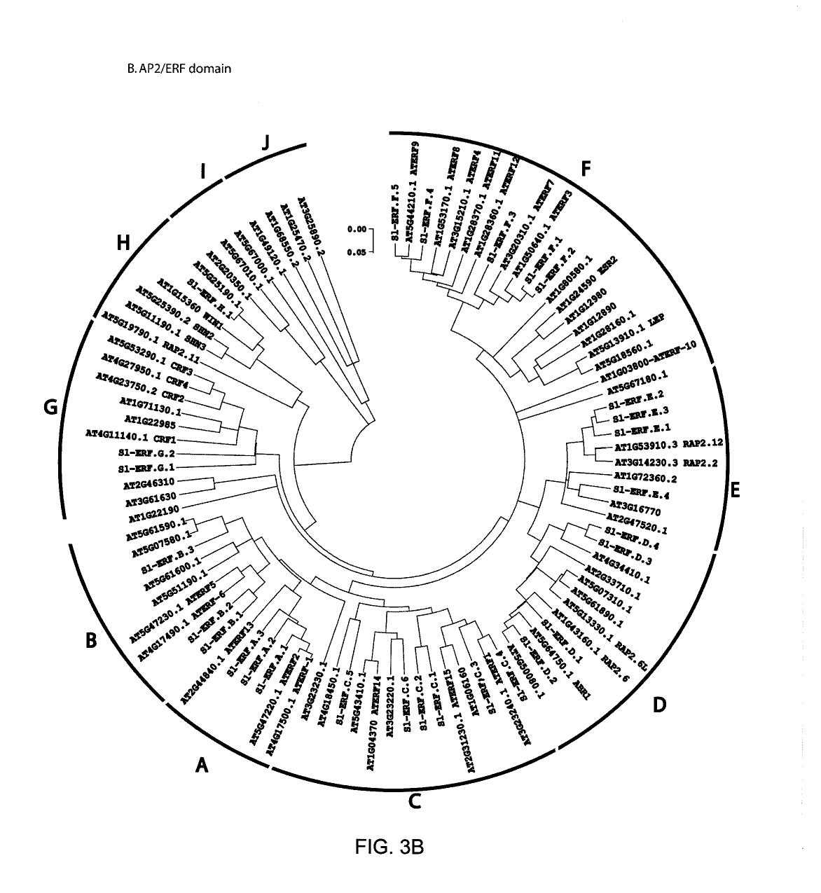

Regulation by ERFs of the following pomoters were assayed:
(A) a synthetic promoter gene containing 4XGCC box,
(B) an ethylene-inducible native promoter containing GCC box,
(C) an ethylene-inducible native promoter lacking the GCC box.

1 refers to the activity of the promoters in the presence of native ERF proteins,

2 refers to the activity of the promoters in the presence of ERF proteins fused to the SRDX repressor domain.

FIG. 6: A—Tomato transgenic lines over-expressing the gene encoding ERF. E2
B—Tomato transgenic lines expressing a “repression version” of ERF. B3 compared to a wild type tomato.

FIG. 7: Over-expression line ERF.C.3::SRDX (dominant negative) presents an elongated shape compared to wild-type (WT). A. Fruit shape is assessed by the ratio width/height for the 3 tested independent lines and the WT. B. Fruit weight have been measured at stage “breaker +10 days” (BK+10) stage in gramme. Values correspond to the mean obtained from measures on 6 fruits, harvested on 5 plants for each lines. Error bars correspond to the standard deviation. Student test for meaning comparison has been done. Statistic value and p-value are indicated above each bar. Stars indicate a significant difference between transgenic line and WT.

FIG. 8: A. Fruits from over-expression lines ERF.E.1::SRDX (dominant negative) are lighter than WT fruit, without any change of shape indicating a smaller fruit. Fruit weight have been measured at BK+10 stage in gramme. B. Fruit shape index of transgenic lines over-expressing ERF.E.1::SRDX at BK+10. Values correspond to the mean obtained from the measure on 6 fruits of 5 plants for each lines. Error bars correspond to the standard deviation. Student test or Mann-Withney test for meaning comparison has been done according data parameters. Statistic value and p-value are indicated above each bar. Star indicate a significant difference between transgenic line and WT.

FIG. 9: Fruit from ERF.C.3::SRDX and ERF.E.1::SRDX over-expressing lines are more yellow than WT at BK+10, and ERF.E.1::SRDX over-expressed lines are less red than WT at BK+10 stage.

Fruit color has been measured with a chromameter and is indicated with 3 coordinate axes: <<a>> (red colour), <<b>> (yellow color) and L (shine aspect). A. ERF.C.3::SRDX 20/1 et 5/8 over-expressing lines showed a higher “b” than WT. B. ERF.E.1::SRDX 123c/6/1 et 34E/1/1 over-expressing lines show a higher “b” than WT. Values correspond to the mean of chromatic index “b” obtained from the measure on 6 fruits of 5 plants for each lines. C. ERF.E.1::SRDX over-expressed lines are less red than WT at stage “breaker +10 days” (BK+10). Values correspond to the mean of chromatic index “a” obtained from the measure on 6 fruits of 5 plants for each lines.

Error bars correspond to the standard deviation. Student test or Mann-Withney test for meaning comparison has been done according data parameters. Statistic value and p-value are indicated above each bar. Stars indicate a significant difference between transgenic line and WT.

FIG. 10: Fruit from ERF.C.3::SRDX and ERF.E.1::SRDX over-expressing lines are slightly more shiny than WT at BK+10. A. ERF.C.3::SRDX 20/1 et 5/8 over-expressing lines show a higher “L” than WT. B. ERF.E.1::SRDX 123c/6/1 et 34E/1/1 over-expressing lines show a higher “L” than WT. C. ERF.H.1::SRDX over-expressing lines are shiner than WT at BK+10 stage.

Values correspond to the mean of chromatic index “L” obtained from the measure on 6 fruits of 5 plants for each lines. Error bars correspond to the standard deviation. Student test or Mann-Withney test for meaning comparison has been done. Statistic value and p-value are indicated above each bar. Stars indicate a significant difference between transgenic line and WT.

FIG. 11: Fruits from ERF.B.3::SRDX and ERF.F.5::SRDX over-expressing lines are affected on sugar content compared to WT at BK+10. A. ERF.B.3::SRDX 99C/1/1 et 47C/6/1 over-expressing lines show a smaller brix degree than WT indicating that fruits from these lines are less sweet than WT. B. ERF.F.5::SRDX 99H/10/1 et 113E/8/1 over-expressing lines show a higher brix degree than WT indicating that fruits from these lines are sweeter than WT. Values correspond to the mean of chromatic index “L” obtained from the measure on 6 fruits of 5 plants for each lines. Error bars correspond to the standard deviation. Mann-Withney test for meaning comparison has been done. Statistic value and p-value are indicated above each bar. Stars indicate a significant difference between transgenic line and WT.

### EXAMPLES

### Example 1

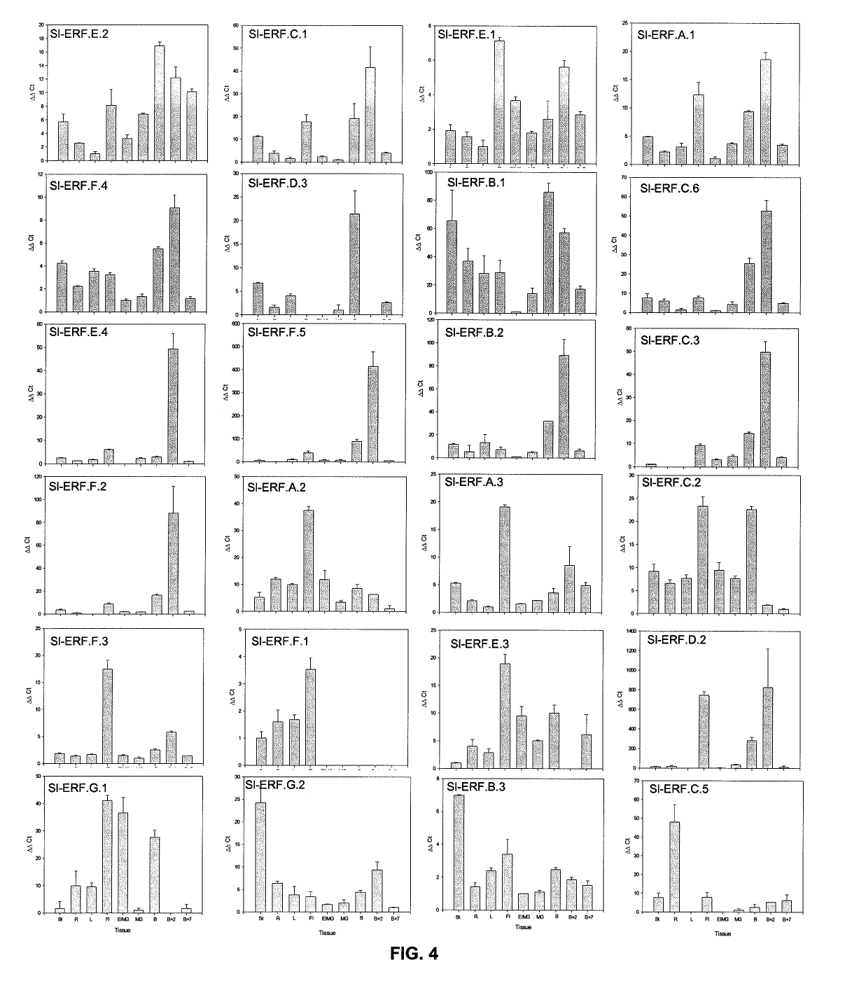

The spatio-temporal pattern of expression of twenty-four members of the tomato ERF gene family was established. Quantitative RT-PCR of ERF transcript in total RNA samples were realized on extracts from Stem (St), Roots (R), Leaves (L), Flower (Fl), Early Immature Green Fruit (EIMG), Mature Green Fruit (MG), Breaker Fruit (B), Breaker +2 days (B+2), and Breaker +7 days (B+7). Results are presented in FIG. 4 and summarized in table 1 below.

| | |
|---|---|
| Mainly expressed in the fruit | A1, B2, C1, C3, C6, D2, D3, E4, F2, F5 |
| Mainly expressed in the | A2, A3, E3, F1, F3, G1 |
| flower organ | |
| Mainly expressed in | C5, G2 |
| vegetative tissues | |
| Constitutive expression | B1, B3, C2, E1, E2, F4. |

These results indicate that ten ERFs are preferentially expressed in the fruit, six ERFs in flower organ and two in vegetative tissues, while the remaining ERFs display a constitutive pattern of expression.

### Example 2

ERF activity on synthetic or native promoter, positioned before a reporter gene, was assessed by transient expression in a single cell system. The fluorescence of the reporter gene was assessed by flux cytometrie. Regulation by thirteen ERFs of the following promoters was assayed:

(A) a synthetic promoter gene containing 4XGCC box,

(B) an ethylene-inducible native promoter containing GCC box, Osmotine promoter,

(C) an ethylene inducible native promoter lacking the GCC box, E4 promoter.

1 refers to the activity of the promoters in the presence of native ERF proteins,
2 refers to the activity of the promoters in the presence of ERF proteins fused to the SRDX domain, a dominant repressing domain.

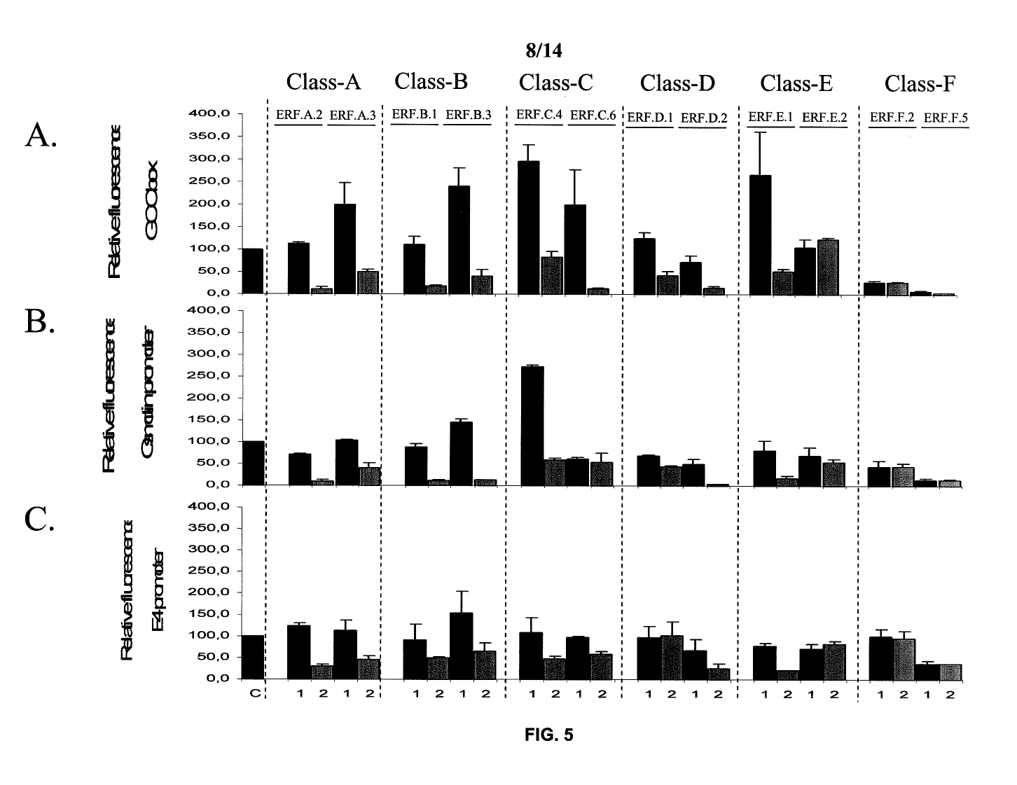

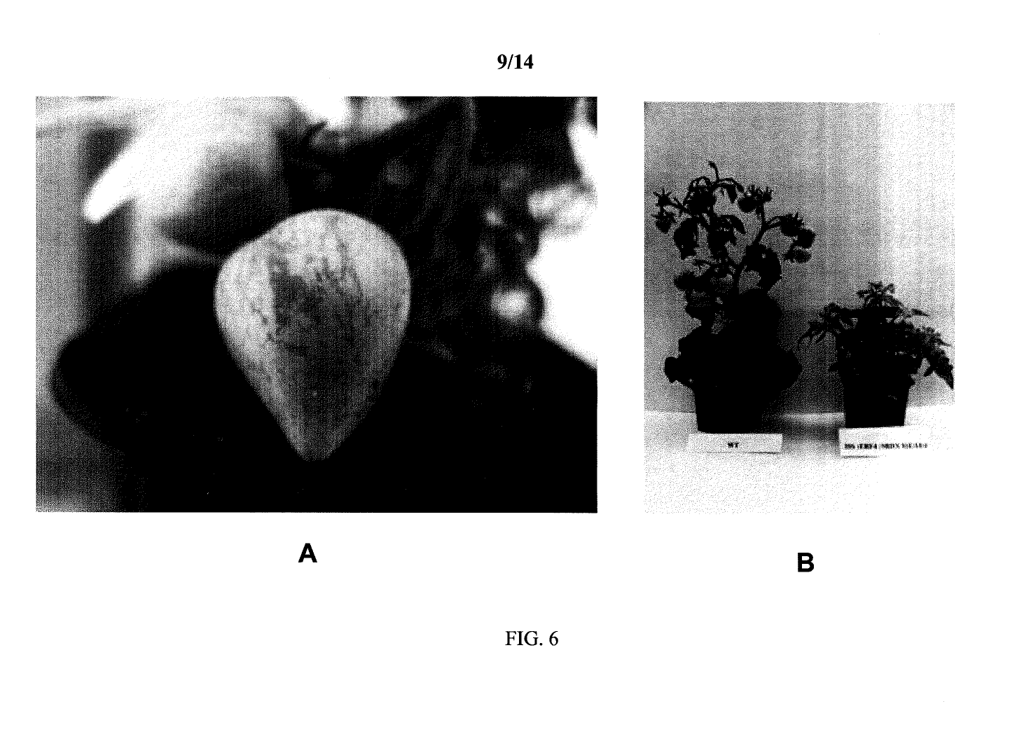

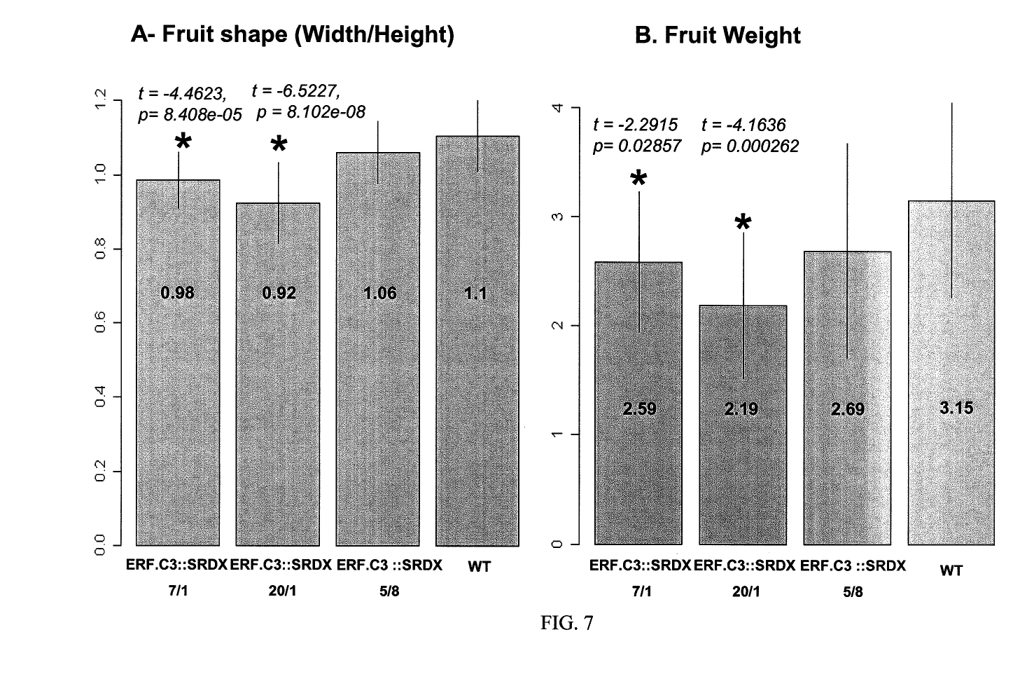

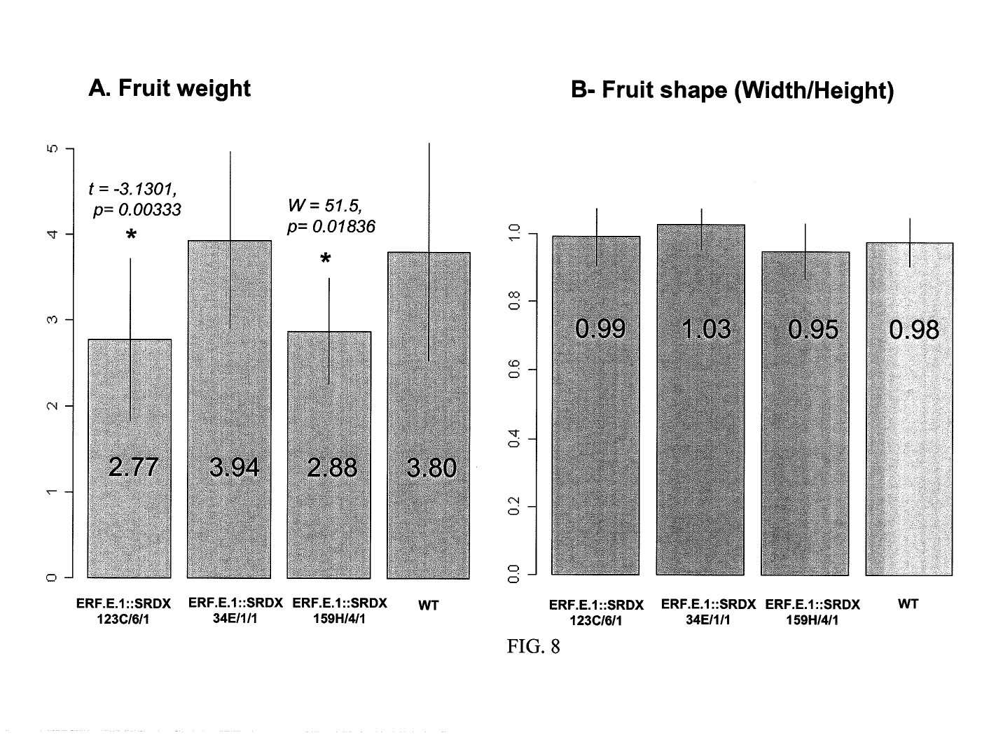

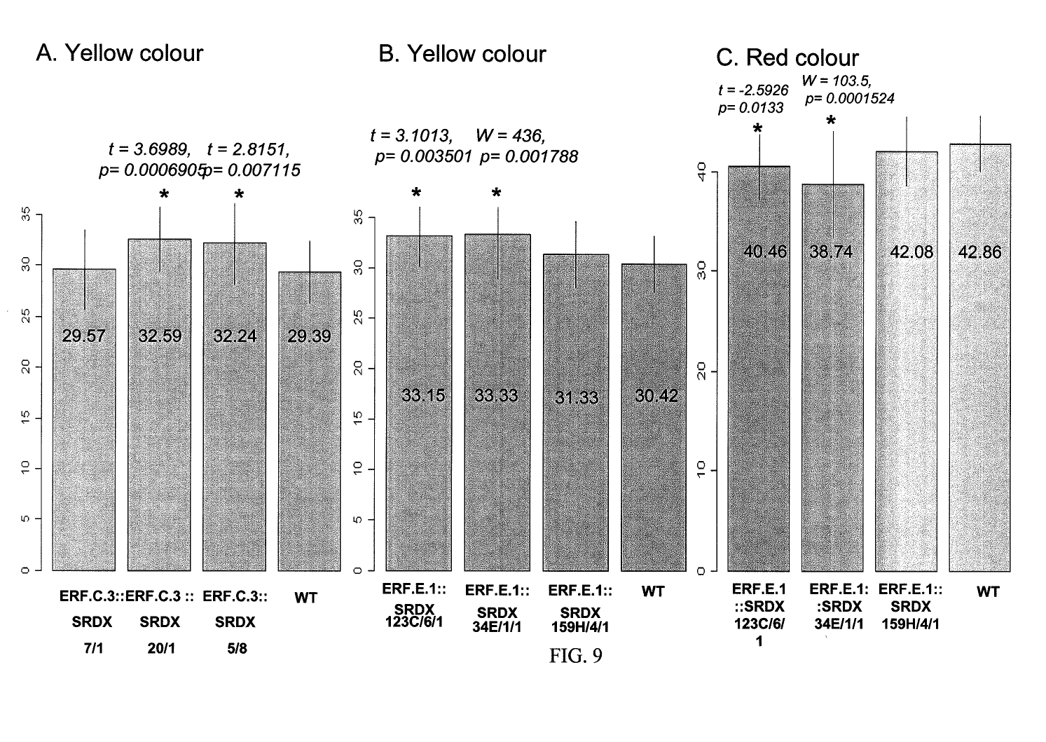

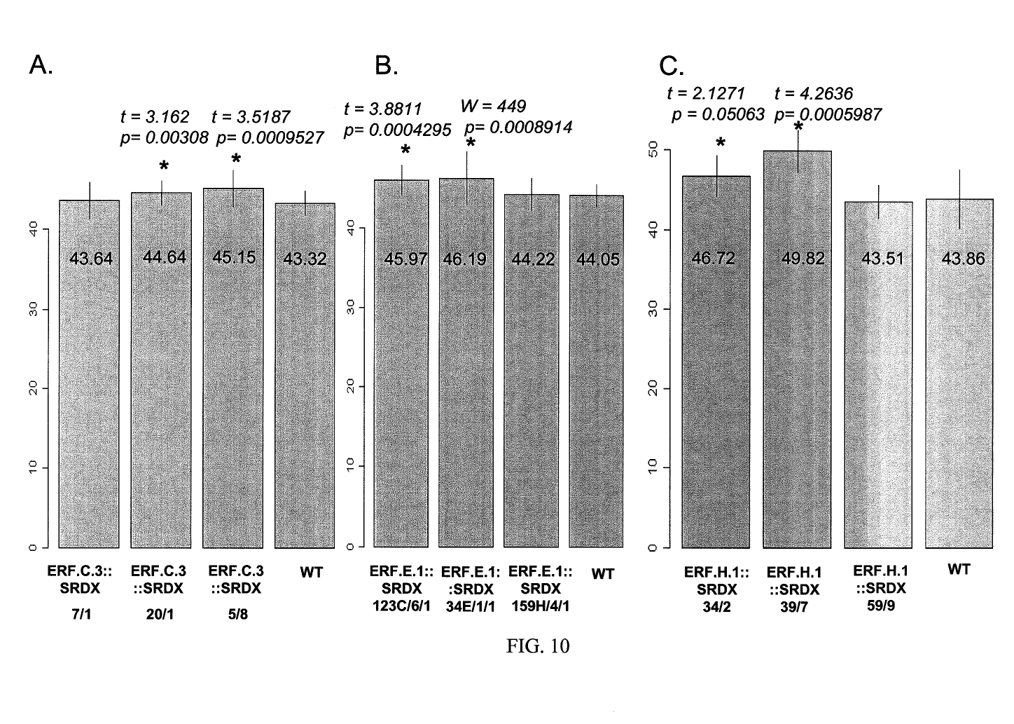

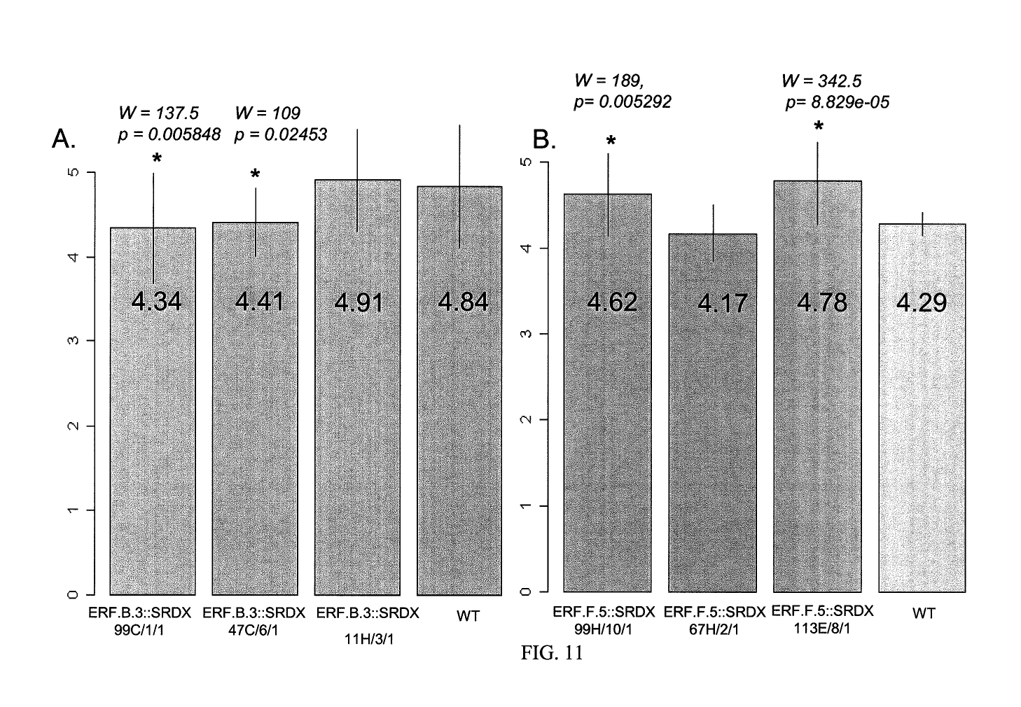

Results are presented in FIG. 5 and in table 2 below:

| | |
|---|---|
| Transcriptional activation of ERF responsive genes | A3, B3, C3, C4, E1 |
| Transcriptional inhibition of ERF responsive genes | F1, F2, F3, F4, F5 |
| ERF able to modulate the activity of ethylene | B3, C3, D2, F5 |
| responsive promoters lacking the GCC box | |

Inventors here show that some ERFs modulate positively or negatively the activity of promoters, and that surprisingly, three ERFs (B3, D2, F5) can also modulate the transcriptional activity of native ethylene-responsive promoters lacking the canonical GCC box (see lane C, E4 promoter,). ERF. C3 shows also this capacity (result not shown).

### Example 3

The physiological effects of the over-expression in tomato plants of the gene encoding ERF. E2 and a “repressing version” of ERF. B3, comprising a SRDX domain such as described below, were investigated. FIG. 6 show that transgenic tomato expressing ERF.E2 (SEQ ID NO 25) have fruits with unusual color (A); and transgenic tomato plant expressing the “repressing version” of ERF.B3 (SEQ ID NO 1) is smaller than the corresponding wild-type tomato plant shown on the left (B).

This experiment demonstrates the impact of ERF expression on different properties of tomato plants.

### Example 4

### Observed Phenotypes in Tomato Lines Expressing a Repressing Version of Six Specific ERFs

The SRDX domain is a modified version of the EAR domain (ERF Amphiphilic Repression domain) naturally present in the ERF sequences of the class F; in the repressor domain of the Aux/IAA transcription factor and in the SUPERMAN transcription factor. A transcription factor (TF) to which the SRDX domain is fused (TF::SRDX) acts as a strong dominant repressor and suppresses the expression of the target genes over the activity of endogenous and functionally redundant transcription factors. (Hiratsu et al., 2003: Hiratsu, K., Matsui, K., Koyama, T., and Ohme-Takagi, M. Dominant repression of target genes by chimeric repressors that include EAR motif, a repression domain, in Arabidopsis. The Plant J. 34, 733-739.). The amino acid sequence of the SRDX domain is the following: GLDLDLELRLGFA.

Results presented below correspond to the analysis of transgenic lines overexpressing a dominant negative repressor chimeric construct. These constructs were obtained by fusion of SRDX domain to each ERF on its C-terminal region. Each transgene (ERF::SRDX) is driven by 35S promoter which is a strong constitutive prokaryote promoter. Each construct was cloned on pBCKH plasmid.

Solanum lycopersicon cv. Microtom has been transformed with pBCKH vector including the chimeric construct (35S::ERF::SRDX). The first transformants were selected on medium containing hygromycine antibiotic. Phenotyping have been done on homozygote plant obtained by selection on medium containing hygromycine. A transgenic line was considered as homozygote if 50 seeds from this plant germinate on selective medium and if the 50 seedlings grow normally on the same selective medium. Once homozygote lines have been isolated, first molecular characterizations have been done. Over-expression of the transgene was checked by RT-PCR, using gene specific primer, on each phenotyped line.

For each chimeric constructs, three clones were tested and compared with a wild-type (WT) line. Results are presented in FIGS. 7 to 11 and are summarized in the table below.

| | | Fruit form, | Sugar | Ethylene |
|---|---|---|---|---|
| Code | | size or color | content | response and |
| name | SEQ ID N^{o} | modifications | modification | production |
| ERF.A.3 | SEQ ID N^{o} 6 | | | Yes |
| ERF.B.3 | SEQ ID N^{o} 1 | | Yes | |
| ERF.C.3 | SEQ ID N^{o} 5 | The fruit is more | | |
| | | elongated with a | | |
| | | smaller size and | | |
| | | the color and | | |
| | | shine are | | |
| | | modified | | |
| ERF.E.1 | SEQ ID N^{o} 4 | Modified size | | |
| | | color and | | |
| | | shine of the fruit | | |
| ERF.F.5 | SEQ ID N^{o} 2 | | Yes | |
| ERF.H.1 | SEQ ID N^{o} 3 | Shine of the fruit | | |
| | | is modified | | |

1. A tomato transcription factor of the Ethylene Responsive Factor family (ERF), having an amino acid sequence selected from the group consisting of
a protein having an amino acid sequence as shown in one of SEQ ID NO 1 to NO 28,
a variant thereof,
a functional fragment thereof and
a functional homologous sequence thereof.

2. An ERF of claim 1 involved in the regulation of the sugar level in the fruit, wherein the ERF has an amino acid sequence as shown in SEQ ID NO 1 or in SEQ ID NO 2, or a variant thereof, or a functional fragment thereof or a functional homologous sequence thereof.

3. An ERF of claim 1 involved in the regulation of the shininess of the fruit, wherein the ERF has an amino acid sequence as shown in SEQ ID NO 3 or a variant thereof, or a functional fragment thereof or a functional homologous sequence thereof.

4. An ERF of claim 1 involved in the regulation of the size of the fruit, wherein the ERF has an amino acid sequence as shown in SEQ ID NO 4 or a variant thereof, or a functional fragment thereof or a functional homologous sequence thereof.

5. An ERF of claim 1 involved in the regulation of the shape of the fruit, wherein the ERF has an amino acid sequence as shown in SEQ ID NO 5 or a variant thereof, or a functional fragment thereof or a functional homologous sequence thereof.

6. An ERF of claim 1 involved in the regulation of the color of the fruit, wherein the ERF has an amino acid sequence as shown in SEQ ID NO 4 or in SEQ ID NO. 5, or a variant thereof, or a functional fragment thereof or a functional homologous sequence thereof.

7. An ERF of claim 1 involved in the response of the fruit to ethylene, or in the production of ethylene, wherein the ERF has an amino acid sequence as shown in SEQ ID NO 6 or a variant thereof, or a functional fragment thereof or a functional homologous sequence thereof.

8. The ERF of claim 1, wherein said factor acts positively or negatively on the transcription of responsive genes.

9. The ERF of claim 1, wherein said protein is encoded by a gene localized on the genome close to at least one specific genetic marker (QTL).

10. A plant cell having a genetic alteration in the regulatory or in the coding sequence of a gene encoding an ERF protein as claimed in claim 1.

11. A plant cell having a genetic alteration in the regulatory or in the coding sequence of a gene encoding an ERF protein, wherein expression of at least one ERF as claimed in claim 1 is enhanced.

12. A plant cell having a genetic alteration in the regulatory or in the coding sequence of a gene encoding an ERF protein, wherein expression of at least one ERF as claimed in claim 1 is partially or totally inhibited.

13. A plant cell having a genetic alteration in the regulatory or in the coding sequence of a gene encoding an ERF protein, wherein the transcriptional activity of at least one ERF as claimed in claim 1 is partially or totally inhibited.

14. A plant containing comprising at least one cell as claimed in Of claim 10.

15. The plant of claim 14, wherein said plant has an increased tolerance to viral, bacterial and/or fungal infections and/or an increased tolerance to insects attacks and/or an increased tolerance to nematodes and/or an increased tolerance to abiotic stresses and/or modifications of fruit properties, and/or of plant agronomic properties, and/or of seed properties.

16. A plant comprising at least one cell having a genetic alteration in the regulatory or in the coding sequence of a gene encoding an ERF protein as claimed in claim 2, wherein the plant cell has a genetic alteration in the regulatory or in the coding sequence of a gene encoding the ERF protein, and wherein said plant shows modifications of the sugar content in the fruit.

17. A plant comprising at least one cell having a genetic alteration in the regulatory or in the coding sequence of a gene encoding an ERF protein as claimed in claim 3, wherein the plant cell has a genetic alteration in the regulatory or in the coding sequence of a gene encoding the ERF protein, and wherein said plant shows modifications in the shininess of the fruit.

18. A plant comprising at least one cell having a genetic alteration in the regulatory or in the coding sequence of a gene encoding an ERF protein as claimed in claim 4, wherein the plant cell has a genetic alteration in the regulatory or in the coding sequence of a gene encoding the ERF protein, and wherein said plant shows modifications of the fruit size.

19. A plant comprising at least one cell having a genetic alteration in the regulatory or in the coding sequence of a gene encoding an ERF protein as claimed in claim 5, wherein the plant cell has a genetic alteration in the regulatory or in the coding sequence of a gene encoding the ERF protein, and wherein said plant shows modifications of fruit shape.

20. A plant comprising at least one cell having a genetic alteration in the regulatory or in the coding sequence of a gene encoding an ERF protein as claimed in claim 6, wherein the plant cell has a genetic alteration in the regulatory or in the coding sequence of a gene encoding the ERF protein, and wherein said plant shows modifications of fruit color.

21. A plant comprising at least one cell having a genetic alteration in the regulatory or in the coding sequence of a gene encoding an ERF protein as claimed in claim 7, wherein the plant cell has a genetic alteration in the regulatory or in the coding sequence of a gene encoding the ERF protein, and wherein said plant shows modifications of fruit shape, size, shininess or color, or of sugar content in the fruit, or the production or response of fruits to ethylene.

22. The plant cell as claimed in claim 10, wherein the plant is a tomato plant.

23. A seed of a plant comprising cells as claimed in claim 10.

24. A method for obtaining a plant having new phenotypic characteristics, comprising introducing into a plant cell a genetic alteration in the regulatory or in the coding sequence of at least one gene encoding an ERF protein as claimed in claim 1 and regenerating at least one fertile plant comprising said cell.

25. The method of claim 24, wherein the plant being regenerated is further crossed with another plant and seeds are harvested, said crossing step being eventually repeated at least one time.
